(19) United States
(12) Patent Application Publication
(10) Pub. No.: US 2017/0038295 A1
(43) Pub. Date: Feb. 9, 2017

(54) COLORIMETRIC SENSOR WITH AUTOMATED READOUT

(71) Applicant: President and Fellows of Harvard College, Cambridge, MA (US)

(72) Inventors: Joanna AIZENBERG, Boston, MA (US); Ian BURGESS, Toronto (CA); Natalie KOAY, Somerville, MA (US); Meredith DUFFY, Somerville, MA (US); Theresa KAY, South Stoney Creek (CA); Elijah SHIRMAN, Arlington, MA (US); Navid ABEDZADEH, Vaughan (CA)

(21) Appl. No.: 15/302,389

(22) PCT Filed: Apr. 10, 2015
(86) PCT No.: PCT/US15/25408

§ 371 Date: Oct. 6, 2016

Related U.S. Application Data
(60) Provisional application No. 61/977,728, filed on Apr. 10, 2014.

(51) Int. Cl. G01N 21/25; G01N 21/77; G01N 33/28; G01N 21/78; G02B 1/00

(52) U.S. Cl. CPC G01N 21/251; G01N 21/78; G02B 1/005; G01N 33/28; G01N 21/774; G01N 2021/7779

(57) ABSTRACT

A system and method to measure properties of a liquid, comprising: a colorimetric sensor comprising a photonic structure that displays a first signature upon exposure to a sample liquid that wets at least some of the interior surfaces of the porous photonic structure and displays a signature different from the first signature as the sample liquid evaporates. The system further includes a device to capture changes in the color of the colorimetric sensor; a memory to store data generated by the device; and a processing unit to analyze the data captured by the device. The processing unit compares the data captured by the device with a reference data, wherein the reference data comprises information regarding a time-dependent response of the colorimetric sensor upon exposure to and removal of a first set of predetermined liquids and to output information regarding the sample liquid.

### RELATED APPLICATION

This application claims priority to U.S. Patent Application No. 61/977,728, filed on Apr. 10, 2014, which is hereby incorporated by reference in its entirety.

### STATEMENT OF GOVERNMENT RIGHTS

This invention was made with support from the Air Force Office of Scientific Research Under Grant No. FA9550-09-0669-DOD35CAP. The United States government has certain rights to this invention.

### FIELD OF INVENTION

The present disclosure is directed to a system with automated readout of colormetric sensors. In particular, the system is based on tracking the color changes of colormetric sensors utilizing automated imaging system, algorithm for data analysis and methods for the analysis of various physical and chemical properties of sample liquids using photonic crystals.

### BACKGROUND

Three dimensional (3D) photonic crystals (PCs), materials with a 3D-periodic variation in refractive index, have been the subject of extensive scientific interest. Even when there is not sufficient index-contrast to allow a complete photonic bandgap, PCs display exceptionally bright reflected colors arising from photonic stop gaps in particular crystal directions. Structural colors from PC structures are exhibited in a wide range of biological organisms, and often display dynamic tunability. Infiltration and inversion of porous 3D photonic crystals with materials that are capable of dynamic actuation has produced a broad class of PCs with structural colors that can be dynamically manipulated by various forces, such as mechanical force, temperature, electrostatic/electrochemical forces, and the like. However, the surface properties of these porous structures were more or less uniform throughout the photonic crystal.

U.S. patent application Ser. No. 13/990,324 describes a three-dimensional porous photonic structure, whose internal pore surfaces can be provided with desired surface properties in a spatially selective manner with arbitrary patterns, and methods for making the same are described. When exposed to a fluid (e.g., via immersion or wicking), the fluid can selectively penetrate the regions of the structure with compatible surface properties.

Described herein is a low-cost, portable, and easy-to-use system with increased sensitivity and the dynamic range of the photonic crystals that will allow for rapid on-site identification of key physical and chemical properties of target samples.

### BRIEF DESCRIPTION OF FIGURES AND DRAWINGS

The following figures are provided for the purpose of illustration only and are not intended to be limiting.

FIG. 1 shows a schematic of the system with a colorimetric sensor for automated readout;

FIG. 2 shows controlled disorder in 3D photonic crystals via drying;

FIG. 3 shows evolution of color and scattering of inverse-opal films (IOFs) during drying;

FIG. 4 shows thickness dependence of spectral evolution during drying;

FIG. 5 shows the time evolution of reflectance during drying (3-layer film). (A) Time evolution of reflectance at normal incidence as dodecane dries. (B) Spectra at the five stages defined by total reflectance.

FIG. 6 shows the time evolution of reflectance during drying (4-layer film). (A) Time evolution of reflectance at normal incidence as dodecane dries. (B) Spectra at the five stages defined by total reflectance;

FIG. 7 shows the time evolution of reflectance during drying (5-layer film). (A) Time evolution of reflectance at normal incidence as dodecane dries. (B) Spectra at the five stages defined by total reflectance;

FIG. 8 shows the time evolution of reflectance during drying (6-layer film). (A) Time evolution of reflectance at normal incidence as dodecane dries. (B) Spectra at the five stages defined by total reflectance;

FIG. 9 shows the time evolution of reflectance during drying (7-layer film). (A) Time evolution of reflectance at normal incidence as dodecane dries. (B) Spectra at the five stages defined by total reflectance;

FIG. 10 shows the time evolution of reflectance during drying (8-layer film). (A) Time evolution of reflectance at normal incidence as dodecane dries. (B) Spectra at the five stages defined by total reflectance;

FIG. 11 shows the time evolution of reflectance during drying (9-layer film). (A) Time evolution of reflectance at normal incidence as dodecane dries. (B) Spectra at the five stages defined by total reflectance;

FIG. 12 shows the time evolution of reflectance during drying (10-layer film). (A) Time evolution of reflectance at normal incidence as dodecane dries. (B) Spectra at the five stages defined by total reflectance;

FIG. 13 shows the time evolution of reflectance during drying (11-layer film). (A) Time evolution of reflectance at normal incidence as dodecane dries. (B) Spectra at the five stages defined by total reflectance;

FIG. 14 shows the drying sequence at five Stages through the comparison of normal-incidence reflection spectra of a 9 layer IOF in different liquids;

FIG. 15 shows that measuring time between specific color signatures in an IOF provides volatility information of a liquid;

FIG. 16 shows that measuring time taken to dry is used to add significant specificity to wetting-based indicators;

FIG. 17 shows that the system can be used for gathering compositional information about complex mixtures having two components by measuring relative time between several sets of signatures;

FIG. 18 shows that the system can be used for gathering compositional information about complex mixtures having three components by measuring relative time between several sets of signatures;

FIG. 19 shows the increase in wetting over time for a mixture of acetone and octane;

FIG. 20 shows the optical appearance of partially wet IOFs exposed to 100% ethanol, 90% ethanol and 10% water mixture, 87.5% ethanol and 12.5% water mixture, 85% ethanol and 15% water mixture and air;

FIG. 21 shows a schematic of the automated readout concept using the IOF indicator strip;

FIG. 22 shows the use of image analysis to extract colored areas in an IOF that has been exposed to a liquid;

FIG. 23 shows a housing that normalizes lighting conditions;

FIG. 24 shows the algorithm component used for image cropping, edge detection and chip isolation;

FIG. 25 shows the algorithm component used for thresholding and area detection;

FIG. 26 shown the algorithm component used for color and edge specific tiebreakers;

FIG. 27 shows the drying stage measured by relative reflected intensity from an IOF in three spectral regions;

FIG. 28 shows CIELAB color maps (top row) assigned to the IOF indicators wetted with non-transparent liquid, such as, crude oil, as illustrated here;

FIG. 29 shows packing group assignment for crude oil according to §173.121 class 3,

FIG. 30 shows the initial boiling point (IBP) frequency of Bakken crudes;

FIG. 31A shows W-indicators for colorimetrically distinguishing common clearn room solvents (top), alcohols (middle), fuel types (bottom).;

FIG. 31B shows the numerated readout from an indicator array, illustrating the differentiation of a wide range of organic compounds, mixtures and petroleum products;

FIG. 32A shows the prediction accuracy analysis for a W-Ink array consisting of 6 chemical gradients;

FIG. 32B and FIG. 32C demonstrate that the mechanics of evaporation also couple strongly to color changes in W-Ink;

FIG. 33 shows a schematic of the system including an IOF to be used for identification of the packaging group of crude oil with an accuracy greater than 95%;

FIG. 34 shows a schematic illustrating a system using the automated readout of wetting and drying of the IOF indicator arrays for identifying information about petroleum compounds and ascertaining their hazard classification;

FIG. 35 shows the sorting of refined petroleum compounds using colorimetric wetting responses from two IOF indicators;

FIG. 36 shows the indication of packaging groups of crude oil samples using the same two IOF-indicator array used in FIG. 35;

FIG. 37 shows the indicator response to initial boiling point of partially distilled crude oils;

FIG. 38 shows the time-evolution of spectral signature during crude oil evaporation;

FIG. 39 shows the reproducibility of time-dependent optical response to crude oil during evaporation;

FIG. 40 shows a schematic of how the system can used to detect history of exposures to several stimuli;

FIG. 41 shows that the system can be used to detect previous exposure of the IOF indicators to liquids with specific pH values;

FIG. 42 shows that the system can be used to detect previous exposure of the IOF indicators to light or heat;

FIG. 43 shows that the system can be used to detect previous exposure of the IOF indicators to oxygen;

FIG. 44 shows the Photo-responsive wettability in IOFs with a poly (Disperse Red 1)-co-(acrylic acid) surface functionality; and

FIG. 45 shows the thermally and mechanically responsive wetting and color via pore collapse.

### SUMMARY OF THE INVENTION

In an aspect a system to measure properties of a liquid is described. The system includes a colorimetric sensor including a photonic structure having functional groups on at least some of the interior surfaces of the porous photonic structure; the colorimetric sensor displays a first signature upon exposure to a sample liquid that wets at least some of the interior surfaces of the porous photonic structure and displays a signature different from the first signature as the sample liquid evaporates. The system further includes a device to capture changes in the signature of the colorimetric sensor as a function of time; a memory to store data representing the changes in signature of the colorimetric sensor and a processing unit to analyze the data captured by the device. The processing unit compares the data captured by the device with a reference data, wherein the reference data includes information regarding a time-dependent response of the colorimetric sensor upon exposure to and removal of a first set of predetermined liquids and outputs information regarding the sample liquid based on said comparing the data captured by the device with a reference data.

In some embodiments, the colorimetric sensor includes a photonic structure having at least two regions; each region having different functional groups on at least some of the interior surfaces of the porous photonic structure.

In some embodiments, the said exposure to the sample liquid includes wetting of the colorimetric sensor with the sample liquid. In some other embodiments, the liquid includes at least two components and the different regions with said different functional groups of the photonic structure attract the components of the liquid differently.

In some embodiments, the signature is a hierarchical defect pattern visible as controlled disorder in the photonic crystal structure that changes as the sample liquid evaporates. In some other embodiments, the signature is color of the colorimetric sensor that changes as the sample liquid evaporates. In some embodiments, the color is detected using bright field images. In some other embodiments, the color is detected using dark field images.

In certain embodiments, the signature is detected using at least one photodetector or photodiode recording total scattering. In some embodiments, the signature is reflectance that change as the sample liquid evaporates. In some other embodiments, the signature is angular distribution of off-angle scattering detected through Bertrand lens images that changes as the sample liquid evaporates.

In some embodiments, the photonic structure is an inverse opal structure, a mesoporous silica, a short range order structure exhibiting structural color, a quasicrystal, or mixtures thereof

In some embodiments, the functional groups includes reactive groups, protecting groups, hydrophilic groups, lyophilic groups, lyophobic groups, nanoparticles, or mixtures thereof.

In some embodiments, the regions having different functional groups are distributed laterally in the photonic crystal. In some other embodiments, the regions having different functional groups are distributed vertically in the photonic crystal.

In some embodiments, the photonic structure is functionalized with perylene diimide surface groups. In some other embodiments, the photonic structure is functionalized with spyropyran-terminated surface groups. In some other embodiments, the photonic structure is functionalized with amine-terminated surface groups.

In some embodiments, exposure of the photonic structure to a previous stimuli displays a different time-dependent response of the colorimetric sensor upon exposure to and removal of a first set of predetermined liquids as compared with the information in the reference data. In some embodiments, the prior exposure comprises of exposure to oxygen. In some other embodiments, the prior exposure comprises of exposure to light or heat. In some other embodiments, the prior exposure comprises of exposure to pH or moisture.

In some embodiments, the device, the memory and the processing unit are housed in a mobile telecommunication device.

In some embodiments, the data is in the form of a video. In some other embodiments, the data is in the form of one or more photographs. In some other embodiments, the data in the form of a series of time-lapse photographs.

In some embodiments, the sample liquid is a crude oil. In some other embodiments, the output information regarding the crude oil is a packaging group.

In some embodiments, the liquid is a refined petroleum product.

In some embodiments, the output information regarding the sample liquid is the composition of its components. In some other embodiments, the output information regarding the sample liquid is volatility. In some other embodiments, the output information regarding the sample liquid is flashpoint. In some other embodiments, the output information regarding the sample liquid is boiling point.

In some embodiments, the lighting condition is normalized by placing the colorimetric sensor and the device to capture changes in the color of the colorimetric sensor in a housing.

In an aspect, a method of identifying the properties of a liquid, includes providing a colorimetric sensor including a photonic structure having functional groups on at least some of the interior surfaces of the porous photonic structure; wherein the colorimetric sensor displays a first signature upon exposure to a sample liquid that wets at least some of the interior surfaces of the porous photonic structure and displays a signature different from the first signature as the sample liquid evaporates; and providing a device to capture changes in the signature of the colorimetric sensor as a function of time; providing a memory to store data representing the changes in signature of the colorimetric sensor; providing a processing unit to analyze the data captured by the device; wherein the processing unit compares the data captured by the device with a reference data, wherein the reference data comprises information regarding a time-dependent response of the colorimetric sensor upon exposure to and removal of a first set of predetermined liquids and outputs information regarding the sample liquid based on said comparing the data captured by the device with a reference data.

In some embodiments of the method, the colorimetric sensor includes a photonic structure having at least two regions; each region having different functional groups on at least some of the interior surfaces of the porous photonic structure.

In some embodiments of the method, said exposure to the sample liquid comprises wetting of the colorimetric sensor with the sample liquid. In some other embodiments of the method, the sample liquid has at least one component that evaporates.

In some embodiments of the method, the signature is a hierarchical defect pattern visible as controlled disorder in the photonic crystal structure that changes as the sample liquid evaporates. In some embodiments of the method, the signature is color of the colorimetric sensor that changes as the sample liquid evaporates. In some other embodiments of the method, the color is detected using bright field images. In some other embodiments of the method, the color is detected using dark field images. In some embodiments of the method, the signature is detected using at least one photodetector or photodiode recording total scattering. In some embodiments of the method, the signature is reflectance that change as the sample liquid evaporates. In some embodiments of the method, the signature is angular distribution of off-angle scattering detected through Bertrand lens images that changes as the sample liquid evaporates.

In some embodiments of the method, the data is in the form of a video. In some embodiments of the method, the data is in the form of one or more photographs.

In some embodiments of the method, the sample liquid is a crude oil. In some other embodiments of the method, the output information regarding the crude oil is a packaging group.

In some embodiments of the method, the sample liquid sample is a refined petroleum product.

In some embodiments of the method, the output information regarding the sample liquid is the composition of its components. In some other embodiments of the method the output information regarding the sample liquid is flashpoint. In some other embodiments of the method, the output information regarding the sample liquid is initial boiling point.

In another aspect a method of tracking history of exposure to prior stimuli, includes providing a colorimetric sensor comprising a photonic structure having functional groups on at least some of the interior surfaces of the porous photonic structure; the colorimetric sensor that displays a first signature upon exposure to a predetermined liquid that wets at least some of the interior surfaces of the porous photonic structure and displays a signature different from the first signature as the predetermined liquid evaporates; and wherein, the exposure of the photonic structure to a previous stimuli, changes the first signature displayed upon exposure to the predetermined liquid to a second signature and displays a signature different from the first and second color as the predetermined liquid evaporates; The method further includes, providing a device to capture changes in the signature of the colorimetric sensor as a function of time; providing a memory to store data representing the changes in signature of the colorimetric sensor; providing a processing unit to analyze the data captured by the device; wherein the processing unit compares the data captured by the device with a reference data, wherein the reference data comprises information regarding a time-dependent response of the colorimetric sensor upon exposure to and removal of a first set of predetermined liquids and to output information regarding the prior stimuli based on said comparing the data captured by the device with a reference data.

In some embodiments of the method, the colorimetric sensor comprises a photonic structure having at least two regions; each region having different functional groups on at least some of the interior surfaces of the porous photonic structure.

In some embodiments of the method, the signature is a hierarchical defect pattern visible as controlled disorder in the photonic crystal structure that changes as the sample liquid evaporates. In some embodiments of the method, the signature is color of the colorimetric sensor that changes as the sample liquid evaporates. In some other embodiments of the method, the color is detected using bright field images. In some other embodiments of the method, the color is detected using dark field images. In some embodiments of the method, the signature is detected using at least one photodetector or photodiode recording total scattering. In some embodiments of the method, the signature is reflectance that change as the sample liquid evaporates. In some embodiments of the method, the signature is angular distribution of off-angle scattering detected through Bertrand lens images that changes as the sample liquid evaporates.

In some embodiments of the method, the prior exposure comprises of exposure to oxygen. In some embodiments of the method, the prior exposure comprises of exposure to light or heat. In some embodiments of the method, the prior exposure comprises of exposure to pH or moisture.

In some embodiments of the method, the photonic structure is functionalized with perylene diimide surface groups. In some embodiments of the method, the photonic structure is functionalized with spyropyran-terminated surface groups. In some embodiments of the method, the photonic structure is functionalized with amine-terminated surface groups.

### DETAILED DESCRIPTION

A system for automated readout that allows increased sensitivity and dynamic range of photonic crystal based indicator devices (also referred hereinafter as “W-Ink indicators”) without sacrificing the simplicity and user friendliness of the device is described. The method is a low-cost, portable, and easy-to-use indicators that will allow for rapid on-site identification of key physical and chemical properties of sample liquids with increased accuracy and repeatability. In doing so, the system utilizes a device to capture signatures produced in a photonic crystal upon exposure to and thereafter evaporation of a liquid, a memory to store the captured data and a processing unit to analyze the image to produce an automated readout.

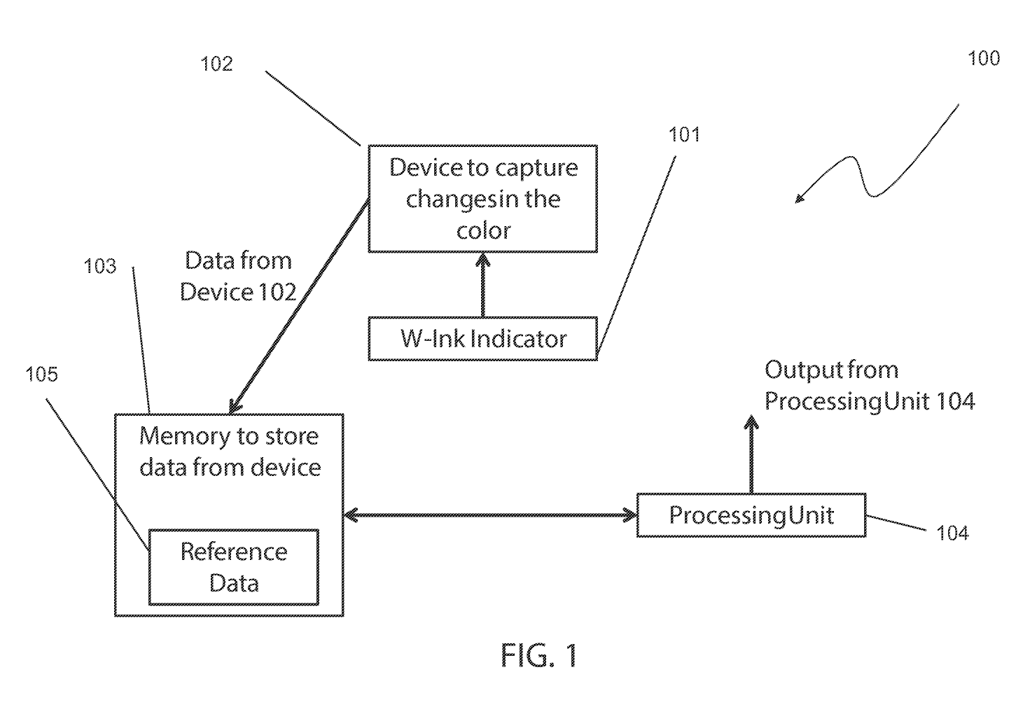

FIG. 1 shows the various components of a system 100 for automated readout that allows expanded increase in the sensitivity and the dynamic range of the photonic crystal based indicator devices. The system includes a W-Ink indicator 101 which produces a static and time-dependent response upon expire to a liquid. The W-Ink indicator 101 is a photonic crystal structure having functional groups on at least some of the interior surfaces of the porous photonic structure and the different functional groups attract with the liquid differently. The system further includes a device 102 to capture changes in the color of the colorimetric sensor when exposed to the liquid. A memory 103 is also included in the system which stores the data generated by the device 102. The system further includes a processing unit 104 to analyze the data captured by the device 102. The processing unit 104 performs a comparison of the data captured by the device 102 with a reference data 105. In some embodiments, the reference data 105 is also stored in the memory 103. The reference data 105 includes information regarding a time-dependent response of the colorimetric sensor 101 upon exposure to and removal of a first set of predetermined liquid. The processing unit 104 uses the comparison of the data captured by the device 102 with the reference data 105 to output information regarding the liquid sample and the accuracy of the match in the comparison.

In certain embodiments, the W-Ink indicator can provide various different types of signatures, such as color information, defect pattern visible as controlled disorder in the photonic crystal structure, reflectance, and the angular distribution of off-angle scattering detected through Bertrand lens images. In some embodiments the W-Ink indicator is a colorimetric sensor having various types of functionality. In some embodiments, the W-Ink indicator includes a single indicator. In some other embodiments, the W-ink indicator includes an array of indicators. Furthermore, in some embodiments, the functionality of the W-ink indicator includes uniform surface chemistry, laterally patterned or continuously varying gradient of surface groups.

In certain embodiments, the W-Ink indicator 101 is a three-dimensional photonic crystal. In certain embodiments, the W-Ink indicator 101 is a three-dimensional photonic crystal having a plurality of interconnected pores. In certain embodiments, the W-Ink indicator is an inverse opal film (“IOF”).

In some embodiments, the W-Ink Indicator has functional groups on at least some of the interior surfaces of the porous photonic structure. In some-other embodiments, the W-Ink Indicator has at least two regions; each region having different functional groups on at least some of the interior surfaces of the porous photonic structure. The W-Ink indicator can displays a first signature upon exposure to a sample liquid that wets at least some of the interior surfaces of the porous photonic structure and displays a signature different from the first signature as the sample liquid evaporates.

In some embodiments, the drying can proceed such that there is an evolution of a hierarchical defect pattern that can be detected as controlled disorder in the photonic crystal structure. In some other embodiments, the color of the W-Ink indicator can change as the liquid evaporates during drying. In some embodiments, the color change of the W-Ink indicator can be detected as a bright field image. In some embodiments the color change of W-Ink indicator can be detected as a dark field image. In some other embodiments, the drying can proceed with a change in reflectance continuously. In some other embodiments, the drying can proceed with a change in the angular distribution of off-angle scattering detected through Bertrand lens images.

In some embodiments, the drying can proceed over multiple stages. For example, the drying can proceed over five distinct stages. These stages can include the following:

Stage 1: disappearance of the over-layer and start of pore-emptying;

Stage 2: Total reflectance decreased to halfway between Stage 1 and the minimum reflectance;

Stage 3: Point of minimum reflectance;

Stage 4: Total reflectance recovered halfway from the minimum value toward the end (dry) value;

Stage 5: Drying completed.

In some embodiments the minimum reflectance, typically observed at Stage 3, depends on the thickness or the number of layers in the photonic crystal structure.

In some embodiments, during the drying process, the reflection also qualitatively changes from that of a uniform thin film (completely wet state), with multiple interference peaks of roughly equal height, to a perfect photonic crystal with finite thickness (empty state), displaying a prominent Bragg resonance alongside smaller side peaks (the latter a result of the finite thickness).

In some embodiments the device 102 is a smartphone camera. In some other embodiments, the device 102 is a single photodetector (e.g. photodiode) measuring total reflectance or scattering. In some other embodiments, the device 102 is an array of photodetectors measuring scattering in different locations or at different angles. In some other embodiments, the device 102 is an array of photodetectors measuring reflectance through color filters (e.g. low-pass, high-pass, band-pass, etc.). In some other embodiments, the device 102 is one or more miniature spectrometers measuring scattering spectrum at one or more angles. In some other embodiments, the device 102 is a camera or imaging device with a Bertrand lens or other means to image scattering angularly. In some embodiments, the device 102 is placed in a housing that normalizes lighting conditions. In some embodiments, color filters can be used for a variety of purposes, such as for monitoring the reflectance during drying.

In some embodiments, the device 102 can capture certain signatures from the W-Ink indicators in the form of data that can be further analyzed. In certain embodiments, photographs and/or reflection spectra of a colorimetric sensor can be obtained. In some embodiments, a single photograph of the indicator can be taken at one time point, e.g. right after exposure to a liquid, or a series of images or a movie can be taken so that the signature can be recorded over time to track the time-evolution of the signature. In some embodiments, a single photodetector measures the reflectance or scattering statically or sequentially over time. In some other embodiments an array of photodetectors measures the scattering in different locations or at different angles statically or sequentially over time.

In some embodiments, the data can be stored into a memory device. In some embodiment, the memory device may be removable. Some exemplary memory device include hard drive, thumb drive, magnetic disk, an optical disk, and magnetic tape. The memory device can further store reference data that can be used as a baseline for comparison. The memory device may also be used for storing software, computer algorithms, and temporary files created by the processing unit during analysis of the data from the device 102. The reference data can be stored in the form of images, time-evolution spectra, static or time-dependent photodetector signals, or static or time-dependent scattering spectrum at one or more angles.

In some embodiments, a processing unit can analyze the measured signatures and report the results (e.g. composition or relevant properties of an unknown liquid) of the analysis to the user. In certain embodiments, the measured data from device 102 can be compared against reference data 105 to determine the chemical composition of a mixture of liquids, the volatility of a liquid mixture, the volatility of the constituents of a liquid mixture, the initial boiling point of a liquid mixture, such as crude oil, the packaging group for a batch of crude oil, the chemical composition of refined petroleum products, and the history of exposure of the W-Ink indicator to prior stimuli, such as moisture, pH, oxygen, light, or heat. This processing unit can also allow the reporting a degree of uncertainty to the user as well as list several possible matches with their relative probabilities in cases where the analysis does not yield a definitive match against the reference data. In some embodiments, the processing unit can provide a user with a probability that a given liquid product (e.g., perfume of a particular brand) is authentic. In some other embodiments, color space analysis enables color differentiation and accounting for colored liquids. In some other embodiments, spectral or overall scattering analysis is used.

In some other embodiments, the processing unit can further carry out additional data manipulation before and/or after comparing the data from device 102 against the reference data 105. For example, the processing unit can carry out for image cropping, edge detection and chip isolation. In some other embodiments, the processing unit can carry out thresholding, area detection, or the like. In some other embodiments, the processing unit can determine tiebreakers for color and/or edge determinations. In some embodiments, the processing unit further includes software such as, image analysis, statistical analysis, comparison with stored calibration curves etc., can be used to analyze these signatures and rapidly print out a meaningful response to the user.

This type of automated readout enables complex information to be extracted from one or more colorimetric sensors. In some embodiments, the system is used to identify properties of complex mixtures. In an exemplary embodiment, the system is used for identification of hazards of crude oil (e.g. flash point, boiling point, explosiveness, etc.). However, the system could be applied to enable identification of properties of many other types of liquids. In some embodiments the properties of the liquids that are evaluated are, but not limited to, refined petroleum products, commercial chemicals, biological fluids, etc.

In another embodiment, when known liquids are used, the system in accordance with this disclosure could be used to determine the history of stimuli that acted on an array of responsive surface groups and report complex and detailed information about prior tampering, in applications where W-Ink film(s) as used as an tamper-indicating device.

In some embodiments, the system 100 can be used to measure time between specific color signatures in a photonic crystal structure to provide volatility information of a liquid. Since the mechanics of evaporation of liquids also couple strongly to color changes in the W-Ink indicator, this provides an extra independent dimension of information on the unknown liquids. In some embodiments the system 100 is used for measuring time taken to dry to add significant specificity to wetting-based indicators and increase the prediction accuracy.

In some embodiments, the system 100 can be used for gathering compositional information about complex mixtures having at least two components by measuring relative time between several sets of signatures.

In some embodiment, the automated readout of wetting and drying of the photonic crystals in system 100 is used to extract compositional information about petroleum compounds and determine their transportation hazard classification. In some embodiments, the system 100 is used to characterize refined petroleum compounds. In some embodiments, the photonic crystal structure has a vertical functionalization gradient with 1H,1H,2H,2H-tridecafuorooctyl-silyl (13F5) as the first functionality and with n-decyl-silyl (DEC) and 3,3,3-trifluoropropyl-silyl (3F) groups respectively as the second functionality.

In some embodiments, in addition to identification of liquids and their properties, the indicator features (wetting and drying) can also be used to detect—via interaction with specific liquids—the indicator's history of exposure to several stimuli. In some embodiments, the system 100 can be used to detect previous exposure of the photonic crystals to light or heat. In some other embodiments, the system 100 can use used to detect moisture/pH exposure of a sample. In some other embodiments, the system 100 shows that the system can be used to detect previous exposure of the photonic crystals to liquids with specific pH values. In some embodiments, functionalization with amine-terminated surface groups. In some embodiments, photonic crystals are functionalized with spyropyran-terminated surface groups. In some other embodiments, the system 100 can be used to detect previous exposure of the photonic crystals to oxygen. In some embodiments, films functionalized with perylene diimide surface groups. In some embodiments, the devices described herein can be successfully fabricated on decals, and in paints, and deposition onto different types of relevant packaging.

### Examples of Measurements Using the System

As described herein, the system 100 uses the various signatures that are generated when the W-Ink indicator is exposed to a sample liquid and as the sample liquid it evaporates from the photonic crystal structure to reveal information about the sample liquid.

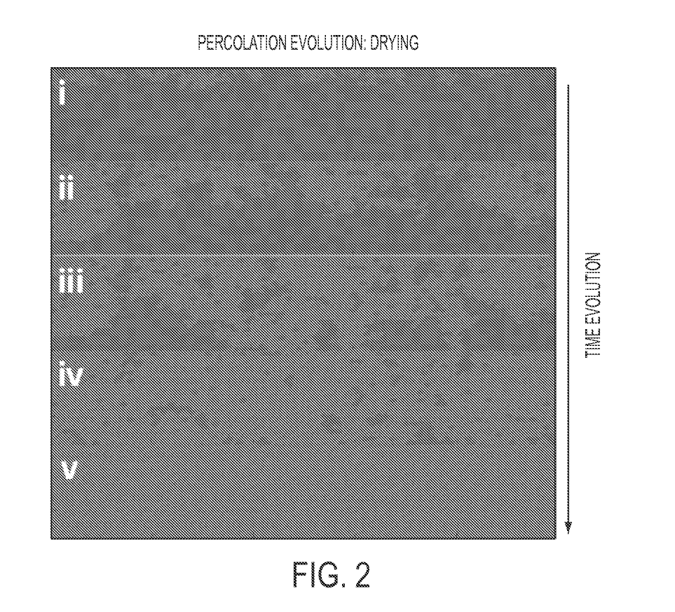

Hierarchical defect patterns can be superimposed on the photonic crystal structure during evaporation of a liquid that has completely imbibed into the porous network. FIG. 2 shows controlled disorder in 3D photonic crystals during drying. As the liquid evaporates from a completely filled IOF, a sequence of hierarchical defect patterns, which evolve over time are formed. FIG. 2 shows the qualitative nature of the photonic structure's evolution (grey=air, black=liquid/matrix) through five time points in a percolation simulation of the sequence of drying from a 2D IOF indicator. As the optical structure evolves from a monolithic thin film, shown in FIG. 2i, representing completely liquid-filled pores, to a perfect photonic crystal, show in FIG. 2ii, representing completely air-filled pores, the degree of disorder first increases as emptying pores invade a predominantly filled structure, as shown in FIG. 2iii, then decreases once empty pores become the majority and filled pores become the defects that recede from the structure, as shown in FIG. 2iv and FIG. 2v, respectively.

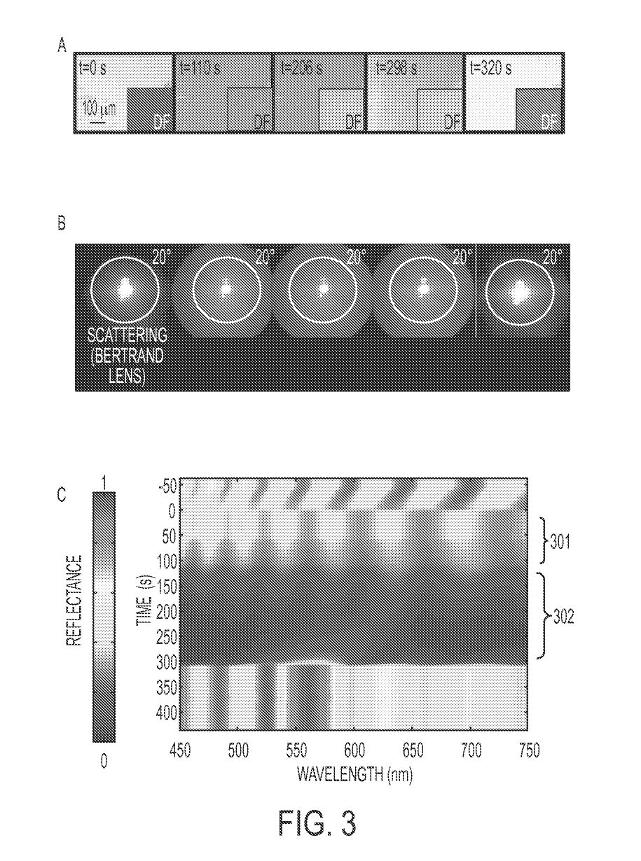

FIG. 3 shows the evolution of color and scattering during drying. To demonstrate drying as shown in FIG. 3, inverse-opal films (IOFs) were functionalized with aliphatic surface groups (n-decyl trichlorosilane). After immersing the functionalized IOFs in alkane liquids, e.g., dodecane, they were flushed under running water. Since water is an immiscible liquid with less affinity for the pore surfaces as compared to alkanes it trapped the alkane liquid inside the pores while removing the alkane on top. After flushing, all excess water slid easily off the surface leaving only the IOF with alkane inside the pores and a very thin liquid film above. This enabled drying to occur concurrently across the entire film, allowing percolation-induced disorder on a large scale to be visualized. FIG. 3A shows the time lapse bright field and dark field (insets) images of an IOF as dodecane evaporates from the pores. FIG. 3B shows the corresponding Bertrand lens images showing angular distribution of scattering. FIG. 3C shows the time evolution of reflectance at normal incidence for a 12-layer IOF. The t=0 denotes the disappearance of the over-layer.

Defect patterns observed during drying evolve continuously over time as liquid evaporate, as seen in FIG. 2 and FIG. 3, with the full spectrum of patterns between complete filling and completely empty pores occurring during the course of each experiment. Similar to the case of partial wetting, fractal-like defect patterns can be superimposed onto the photonic crystal structure as the liquid dries. These patterns can be understood via the theory of invasion percolation. In some embodiments, random variations in the neck geometry make some liquids produce a stronger pinning effect than others on the receding meniscus during drying. As with partial wetting, this underlying sub-wavelength broken symmetry directs the evolution of the hierarchical defect patterns.

At the onset of drying before the pores have emptied, the IOF behaves optically as a homogeneous thin film due to index matching between the structure and fluid, as is evident from the interference fringes in the reflectance spectrum at normal incidence, as seen in FIG. 3C, and the lack of off-angle scattering, as seen in FIG. 3B, i. The fringes blueshift, as seen in region marked 301, as the thickness of the film reduces as the thin over-layer shrinks before the pores begin to empty. The complete evaporation of the over-layer is clearly marked in the reflectance spectrum by an abrupt blueward jump, as seen in region marked 302, in the fringes, as marked in FIG. 3C.

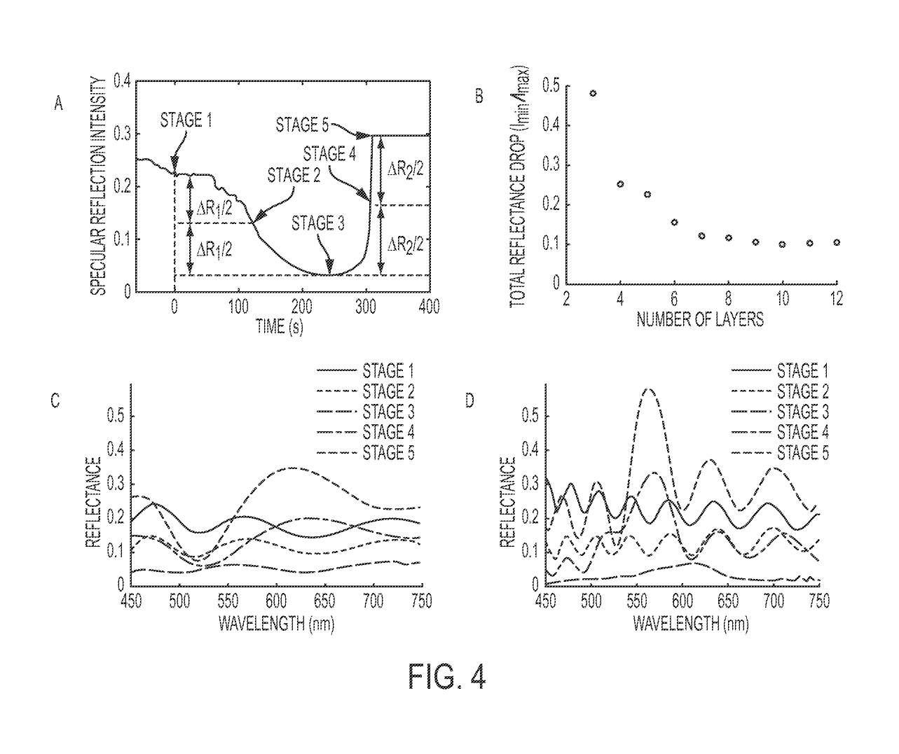

As the drying front invades the structure, scattering from disorder increases until the characteristic percolation length, I_{scat}, is roughly equal to the thickness, h, i.e., l_{scat}˜h, and ˜50% of the pores have emptied. After this the trend reverses as filled pores become the minority (defects) and further evaporation leads to increased order in the structure. This trend is seen in FIG. 3 and FIG. 4.

FIG. 4 shows the thickness dependence of spectral evolution during drying. FIG. 4A shows the time evolution of total reflectance between 450 nm and 750 nm, where t=0 denotes the disappearance of the over-layer. Spectral signatures were compared at five different stages defined by the total reflectance: Stage 1: disappearance of the over-layer and start of pore-emptying; Stage 2: Total reflectance decreased to halfway between Stage 1 and the minimum reflectance; Stage 3: Point of minimum reflectance; Stage 4: Total reflectance recovered halfway from the minimum value toward the end (dry) value; Stage 5: Drying completed. FIG. 4B shows the thickness dependence of the total reflectance change between Stage 3 and 5. FIG. 4C shows the reflectance spectra at all five stages for a 5 layer IOF. Similarly, FIG. 4D shows the reflectance spectra at all five stages for a 12 layer IOF.

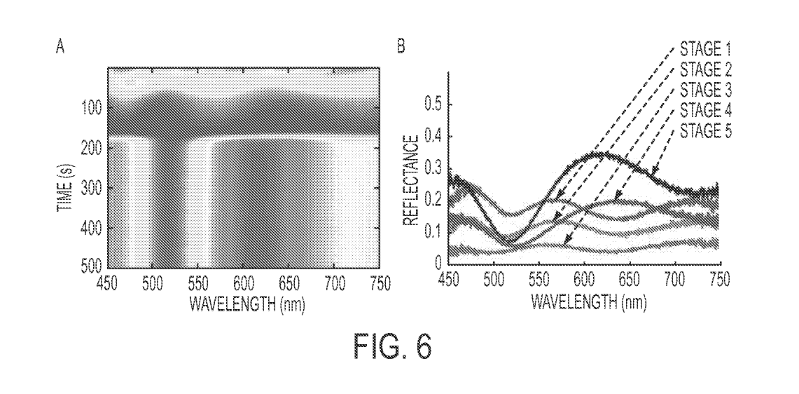

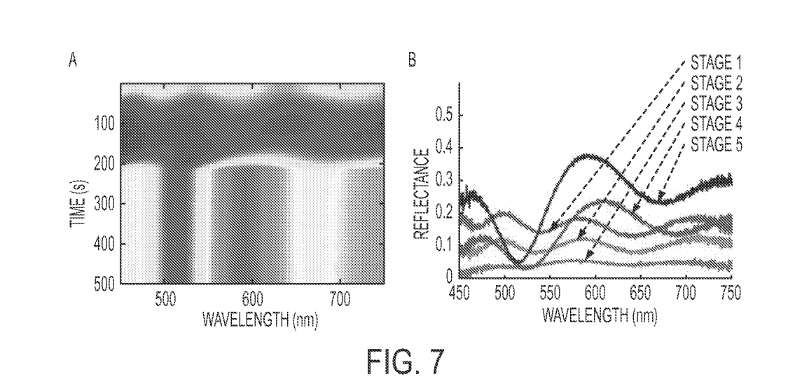

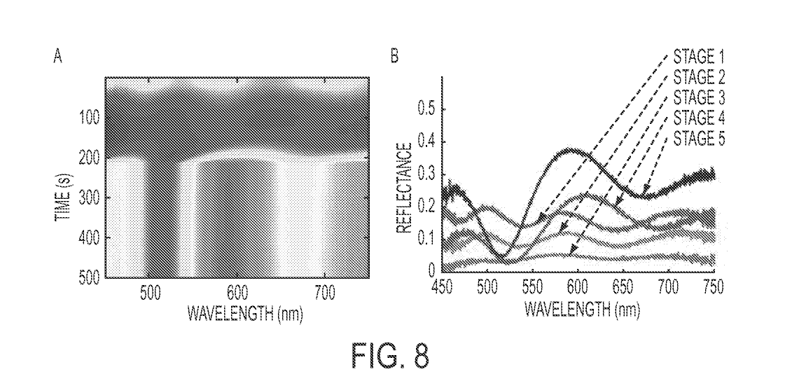

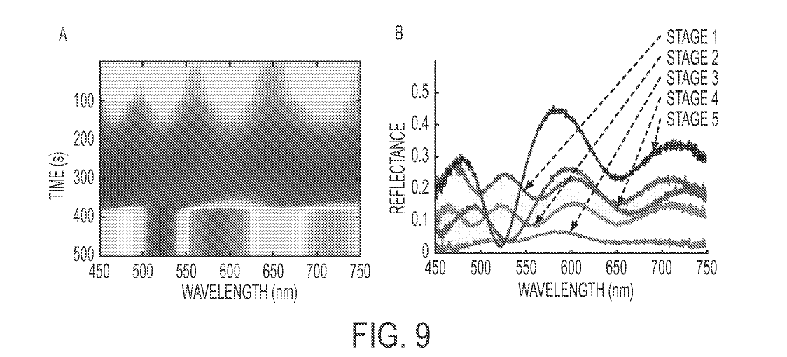

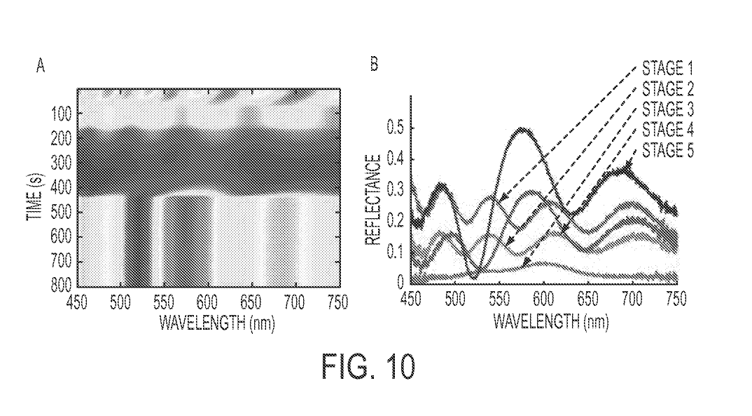

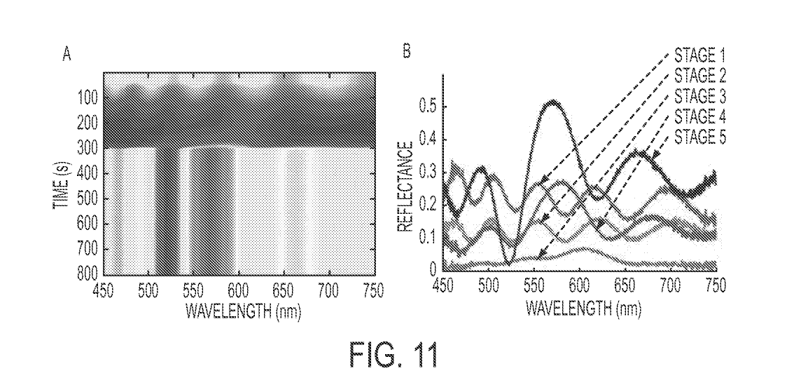

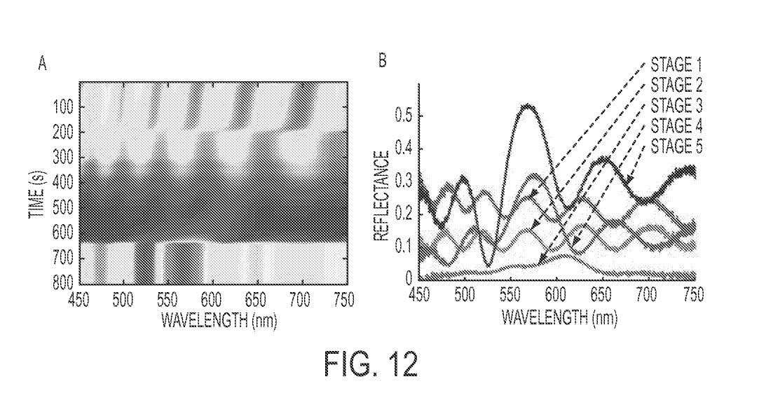

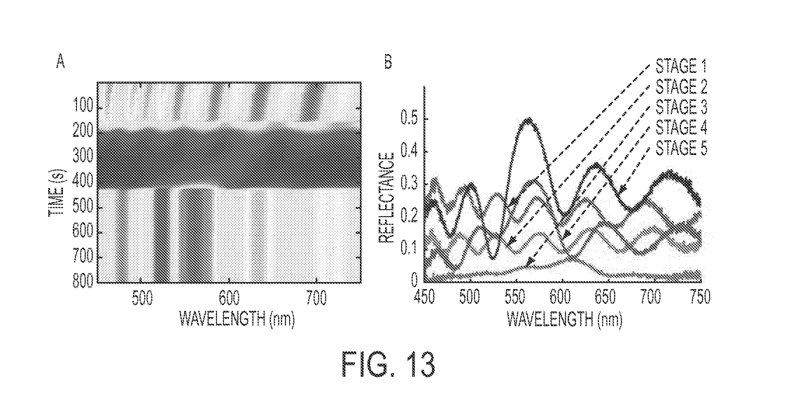

The sequential increase and then decrease of disorder and off-angle scattering is evident from the first increasing then decreasing shift of intensity from the bright field to the dark field images, as shown in FIG. 3A, and the U-shaped time dependence of reflectance at normal incidence, as shown in FIG. 3C and FIG. 4A. The strength of scattering and the suppression of reflectance when it is at its maximum (l_{scat}˜h) increases with increasing thickness of the film. Time-resolved reflection spectra of dodecane evaporation from IOFs having thicknesses form 3 to 12 layers are shown in FIG. 3C and FIG. 5 through FIG. 13. The minimum reflectance is shown as a function of film thickness in FIG. 4B, showing the trend.

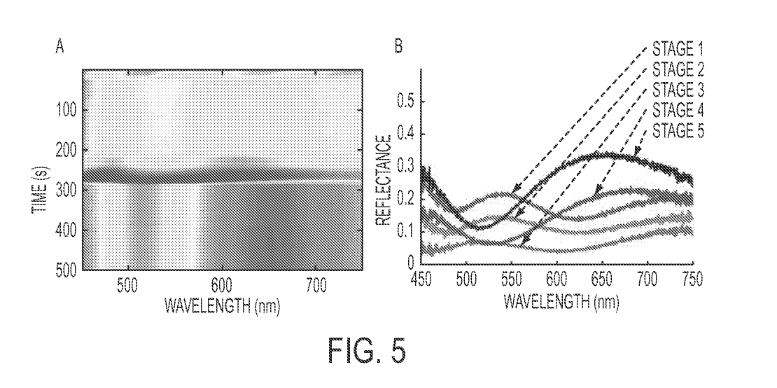

During the drying process, the reflection also qualitatively changes from that of a uniform thin film (completely wet state), with multiple interference peaks of roughly equal height, to a perfect photonic crystal with finite thickness (empty state), displaying a prominent Bragg resonance alongside smaller side peaks (the latter a result of the finite thickness). As evident from FIG. 3C, and FIG. 5 through FIG. 13, the qualitative transition of the reflectance spectrum from one character to the other and the emergence of a Bragg resonance occurs fairly abruptly. Notably this transition occurs after the point of minimum reflectance (maximum disorder) for the thinnest samples), while it occurs before this point for thicker samples. In some embodiments, the qualitative transition of the reflectance spectrum from one character to the other and the emergence of a Bragg resonance occurs after the point of minimum reflectance (maximum disorder) for samples where h<˜7 layers. In some embodiments the qualitative transition of the reflectance spectrum from one character to the other and the emergence of a Bragg resonance occurred before this point for samples where h>8 layers. This is illustrated in FIG. 5 through FIG. 13 and FIG. 4C.

Specifically, FIG. 4C compares the reflectance spectrum for IOFs of 5 layers and 12 layers at five different stages of dodecane drying: (Stage 1) the disappearance of the overlayer and onset of percolation of the drying front into the pores; (Stage 2) the reduction of total reflectance to halfway between Stage 1 and the minimum reflectance; (Stage 3) the point of minimum total reflectance; (Stage 4) the recovery of total reflectance to halfway between the minimum reflectance and a dry film; and (Stage 5) completion of drying. Spectra are also shown for time points 5 s before and 5 s after minimum reflectance (Stage 3). For the IOF containing 5 layers, the spectrum at Stage 3 looks qualitatively the same as for Stages 1 and 2, whereas for 12 layers, a Bragg resonance is prominent by Stage 3, although its peak is redshifted in comparison to the dry peak (Stage 5).

While it is expected that scattering should be maximized when half of the pores have emptied and l_{scat}˜l_{PhC}˜h, it is expected that the qualitative change in the spectrum shape and the appearance of a Bragg resonance depends on when the characteristic photonic crystal grain size reaches a critical value, N, that is independent of h, i.e., l_{scat}˜l_{PhC}˜N. For thin films, where h<N, the point of minimum reflectance occurs before the qualitative transition, whereas the Bragg appearance of a Bragg resonance occurs before the point of minimum reflectance for thicker films, i.e., when h>N. This critical thickness, N, is estimated from the time-resolved spectra in FIG. 3C and FIG. 5 through FIG. 13 as about 6-7 layers for the silica IOFs described here.

FIG. 4 shows the thickness dependence of spectral evolution during drying. FIG. 4A shows the time evolution of total reflectance between 450 nm and 750 nm, where t=0 denotes the disappearance of the over-layer. Spectral signatures were compared at five different stages defined by the total reflectance: Stage 1: disappearance of the over-layer and start of pore-emptying; Stage 2: Total reflectance decreased to halfway between Stage 1 and the minimum reflectance; Stage 3: Point of minimum reflectance; Stage 4: Total reflectance recovered halfway from the minimum value toward the end (dry) value; Stage 5: Drying completed. FIG. 4B shows the thickness dependence of the total reflectance change between Stage 3 and 5. FIG. 4C shows the reflectance spectra at all five stages for a 5 layer IOF. Similarly, FIG. 4D shows the reflectance spectra at all five stages for a 12 layer IOF.

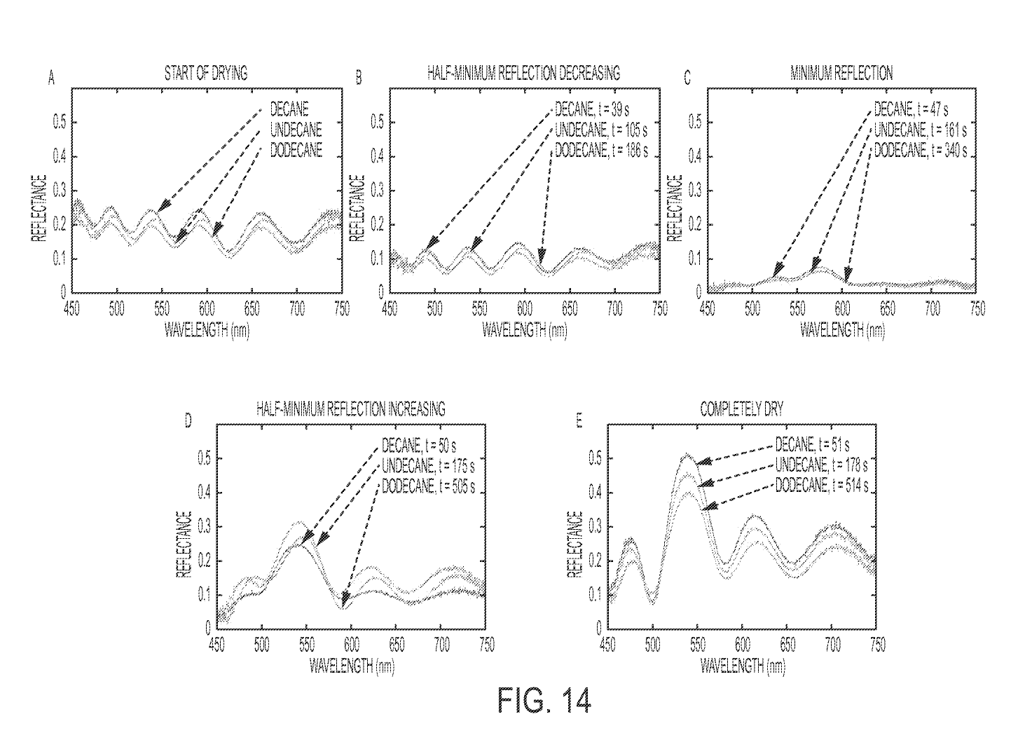

Although the time-scale associated with the drying process increases with the liquid's volatility, the sequence of partially disordered defect patterns and their spectral signatures can be reproduced from one run to the next and across liquids with different volatility. FIG. 14 shows the drying sequence at five Stages through the comparison of normal-incidence reflection spectra of a 9 layer IOF in different liquids, as shown in FIG. 4A. Comparison of normal-incidence reflection spectra of an IOF (9 layers) at the five stages of drying: (A) Stage 1: start of drying; (B) Stage 2: total reflectance decreases to half of the minimum with respect to stage 1; (C) Stage 3: point of minimum total reflectance; (D) Total reflectance recovered to halfway between the minimum and that of a dry film; Stage 5: dry film. While the time taken for the color to evolve between different stages depends on the volatility profile of the liquid, the color at each stage is a function of the structure and the degree of filling, and does not depend strongly on what is filling it. Additionally, while the time taken to reach each stage varies greatly between the liquids, the spectral signatures themselves show relatively little difference. Given the initial condition that the drying front invades from the top of the film, enforced by the manner in which the liquid over-layer was removed with water purging, the order in which the pores empty as an IOF dries is driven predominantly by subtle asymmetries in the pore and neck structure as opposed to depending strongly on properties of the liquid, such as, volatility, surface tension, etc.

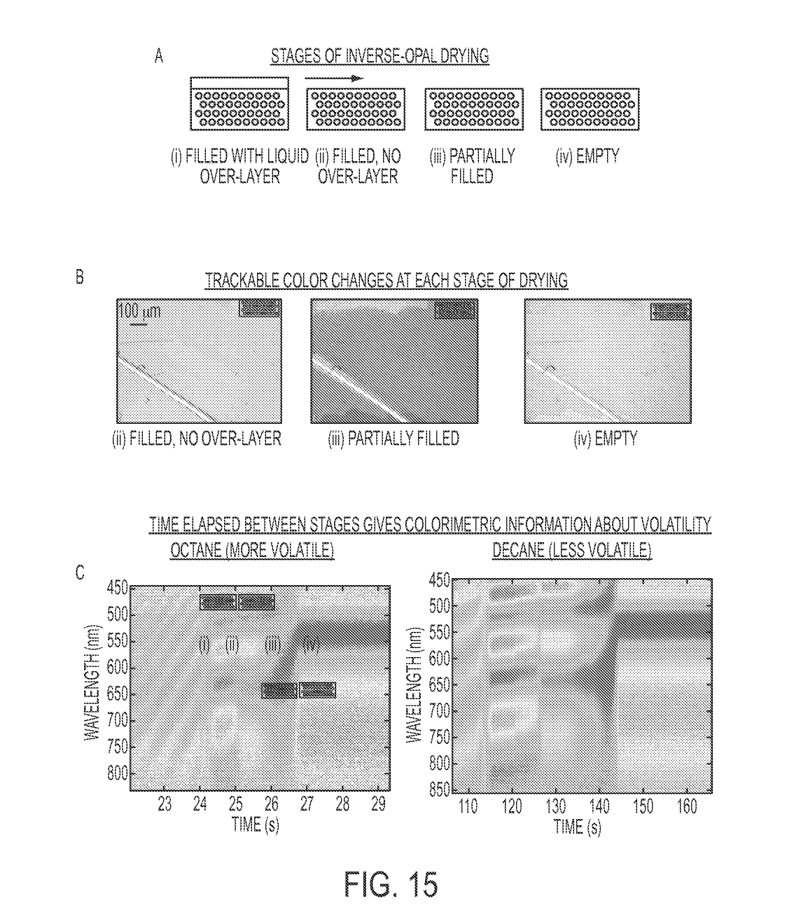

FIG. 15 shows that the system can be used to measure time between specific color signatures in an IOF. This provides volatility information of a liquid. FIG. 15A shows the various stages of the inverse opal drying schematically. FIG. 15Ai shows the IOF filled with liquid along with an over-layer on the top surface. FIG. 15Aii shows the IOF filled with no over-layer. In some embodiments the over-layer is removed by purging with water. FIG. 15Aiii shows a partially filled IOF where part of the liquid has evaporated from the structure. FIG. 15Aiv shows an empty IOF after the drying has completed. FIG. 15Bii, FIG. 15Biii, and FIG. 15Biv shows the trackable color changes at each stage of the drying corresponding to the stages of drying shown in FIG. 15Aii, FIG. 15Aiii, and FIG. 15Aiv, respectively. FIG. 15Bi, corresponding to FIG. 15Ai is not shown here. FIG. 15C shows the time evolution of reflectance for two different liquids with different volatility, such as octane (more volatile) and decane (less volatile). As shown in FIG. 15C the time elapsed between stages of drying gives colorimetric information about the volatility of the liquid.

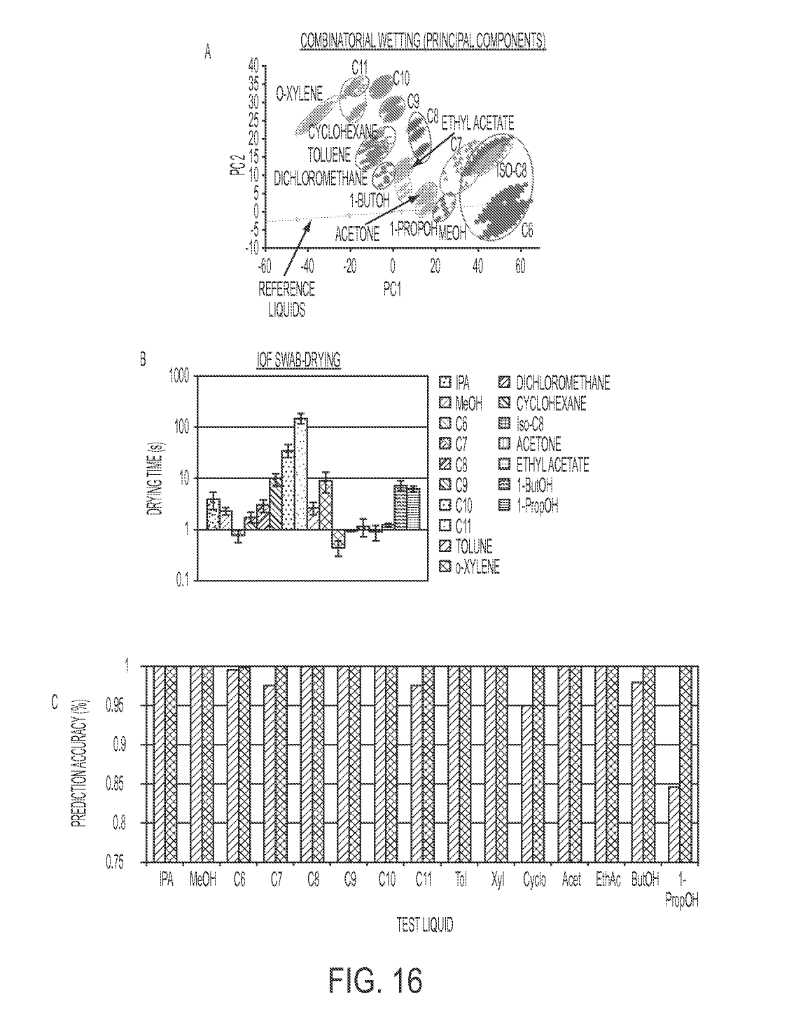

FIG. 16 shows that the system can be used to measure time taken to dry. This information is used to add significant specificity to wetting-based indicators. FIG. 16A shows the prediction accuracy analysis for a IOF or a W-Ink array consisting of 6 chemical gradients. A principal component analysis algorithm was used to determine the accuracy of differentiation between liquids. Comparing each reading to the data set excluding that point, this array identified liquids with 98.6% accuracy from a library of 15 common solvents, as shown in in FIG. 16B and FIG. 16C. The mechanics of evaporation also couple strongly to color changes in the IOF or W-Ink indicator, providing an extra independent dimension of information on the unknown liquids. FIG. 16B shows the measurements of the time to the reappearance of color after swabbing with liquid. FIG. 16C shows the information extracted from the wetting patterns in FIG. 16A. Combining the information in FIG. 16A and FIG. 16B increased the prediction accuracy to 99.99% as shown in FIG. 16C.

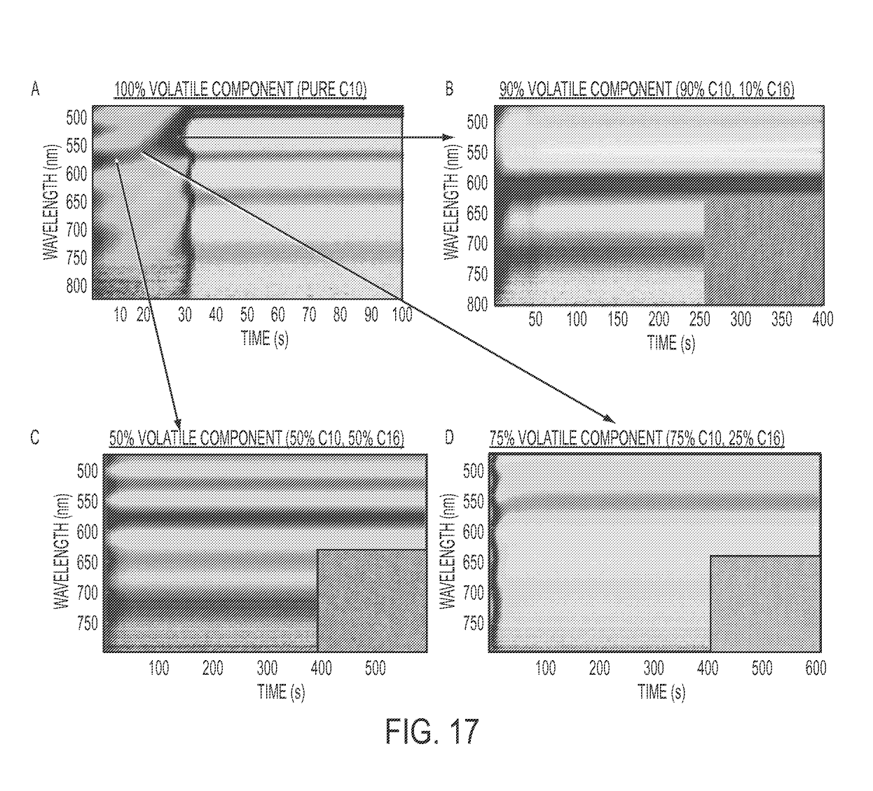

FIG. 17 shows that the system can be used for gathering compositional information about complex mixtures having two components by measuring relative time between several sets of signatures. FIG. 17A shows the complete reflectance evolution during evaporation of pure n-decane (C10). FIG. 17B, FIG. 17C, and FIG. 17D shows the reflectance evolution of mixtures of C10 and hexadecane (C16) in different proportions, with FIG. 17B having 90% C10, FIG. 17C having 50% C10, and FIG. 17D having 75% C10. In each case, evolution of the optical signature rapidly slows down (quasi-static on the time-scales shown) when the C10 component has evaporated. The optical signature at this point provides information about the volatile fraction of the mixture.

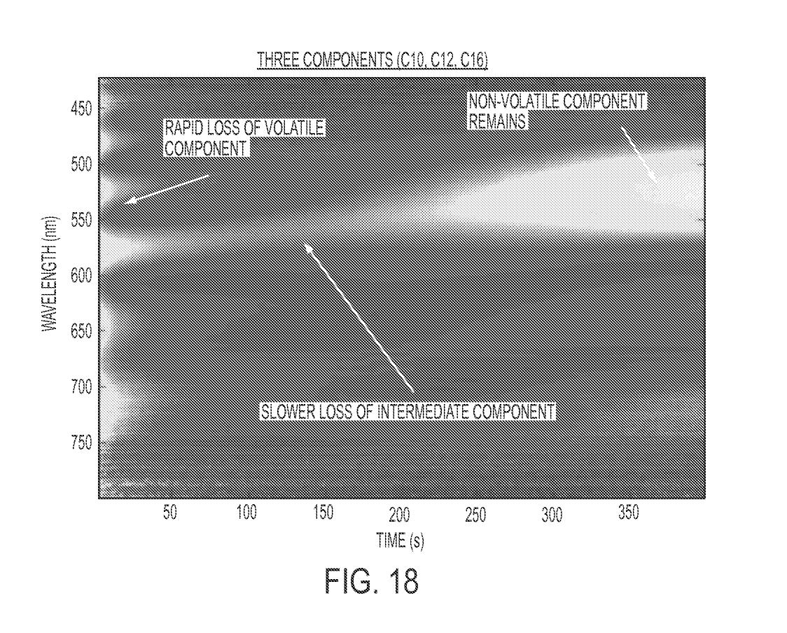

FIG. 18 shows that the system can be used for gathering compositional information about complex mixtures having three components by measuring relative time between several sets of signatures. This reveals compositional information about complex mixtures having three component, pure n-decane (C10), dodecane (C12) and hexadecane (C16). As a multi-component mixture dries, the relative speeds of different stages of evolution of the optical signature allow the different components and their relative concentrations to be discerned. In an embodiment, the reflectance drops very rapidly at the beginning, approaching the point of minimum reflectance, as the most volatile component (C10) evaporates. The evolution then slows down considerably, but continues progressing through minimum reflectance up until the reflectance has recovered roughly halfway to the dry state, at which point the C12 (intermediate volatility) has also evaporated. At this point the evolution stops as only the non-volatile component (C16) remains.

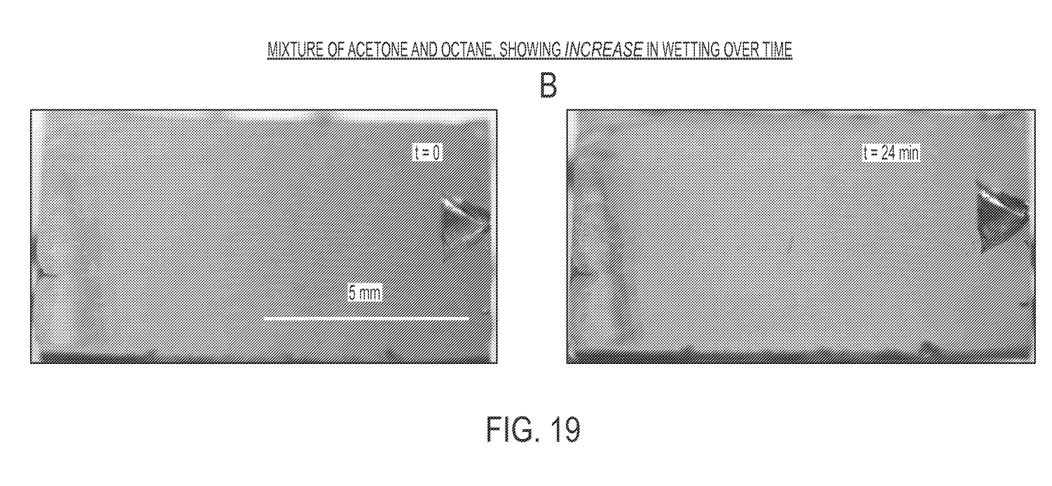

In certain types of mixtures the wettability of the liquid mixture may increase during evaporation, as its composition changes due to more rapid loss of the more volatile components. This wettability increase can occur as a result of the surface tension decreasing as the more volatile component reduces in concentration. FIG. 19 shows the increase in wetting over time for a mixture of acetone and octane. The wettability increases as a result of the surface functionalizations having a greater affinity for the less volatile fractions. When this occurs, the optical signature of the IOF will change over time, reflecting the increasing degree of wetting as evident from the reduction of wetted area at t=24 minutes, as showin in FIG. 19B as compared with the area wetted at t=0 as shown in FIG. 19A.

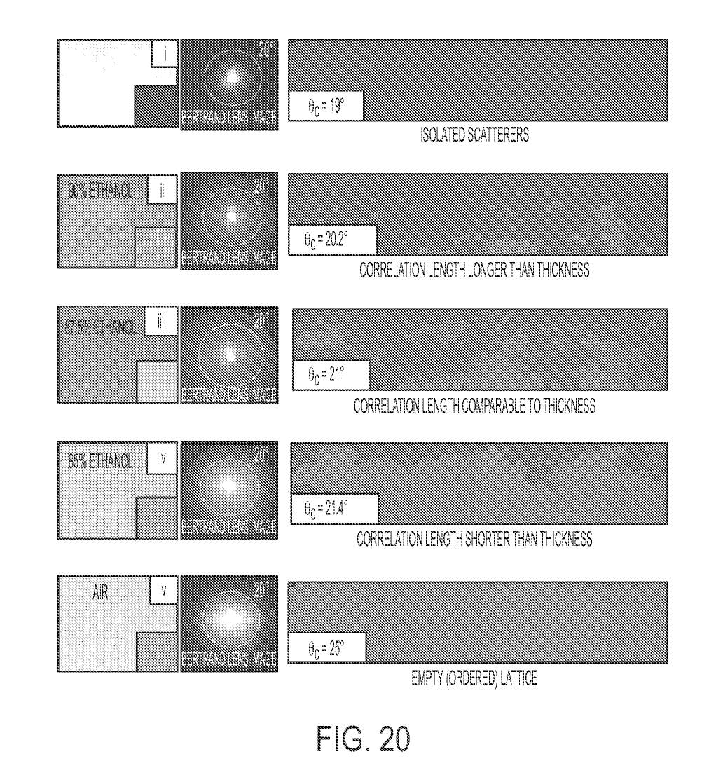

Using machine-discernible assessments of partial wetting enhances the sensitivity of wetting-indicators such as IOFs. FIG. 20 shows the optical appearance of partially wet IOFs exposed to 100% ethanol, 90% ethanol and 10% water mixture, 87.5% ethanol and 12.5% water mixture, 85% ethanol and 15% water mixture and air. The left column of images in FIG. 20 shows the Bright-field and dark field (inset) images of a DEC-functionalized IOF immersed in different ethanol-water mixtures. The center column of images in FIG. 20 shows the corresponding Bertrand lens images showing angular distribution of scattering. The right column of images in FIG. 20 shows filling profiles generated by percolation simulations on a 2D inverse-opal film with a hexagonal lattice. The intrinsic contact angles for each simulation is shown at the bottom of the image. This illustrates that the different types of partial filling patterns produced, qualitatively corresponding to the images on the left; from top, in order of increasing intrinsic contact angle. The right column of image corresponding to FIG. 20i shows that a near completely filled lattice is effectively transparent due to index matching with the Si substrate that acts as a broadband reflector; The right column of image corresponding to FIG. 20ii shows that some pores remain air-filled, but the percolation length is much larger than the film thickness, leading to increased non-specular scattering (darker bright-field and brighter-dark-field images) that is still broadband. The right column of image corresponding to FIG. 20iii shows that maximum non-specular scattering produced when the percolation length is comparable to the thickness and the filling fraction is 50% (darkest bright-field image and brightest dark-field image), with the onset of color also appearing as the photonic crystal grains of a significant size remain. The right column of image corresponding to FIG. 20iv shows percolation depth much shorter than the thickness. Here the non-specular scattering is higher than for a non-wetted lattice, but Bragg scattering is prominent. The right column of image corresponding to FIG. 20v shows a completely non-wetted lattice where strong specular scattering and structural color from Bragg resonance can be seen.

When an IOF is immersed in liquid, the meniscus can move from one pore to the next if the intrinsic contact angle (θ_{c}) is smaller than the re-entrant neck angle (φ₀). In some embodiments, the IOFs contain necks whose neck angles vary randomly according to a fairly narrow distribution, such as, ±3°. It is estimated that most liquids are likely to have θ_{c} far outside the distribution of neck angles, leading to penetration that is either complete or non-existent. However, when IOFs are immersed in liquids whose θ_{c} falls within the narrow range defined by the neck angle distribution, incomplete wetting occurs, as some necks will pin the meniscus while others will not. At equilibrium, the liquid will occupy the network of all paths from the outer surface that are connected by necks that do not pin (φ₀>θ_{c}). Since the refractive-index contrast is diminished in the filled pores, filling has the optical effect of removing them from the structure. Thus, partial wetting imposes fractal-like patterns of defects (missing lattice sites) onto the photonic structure. These patterns of disorder are well described by bond-percolation theory and can be tuned by adjusting θ_{c} for the liquid. This tuning is shown in FIG. 20 using different mixtures of water and ethanol to create a continuum of θ_{c} values.

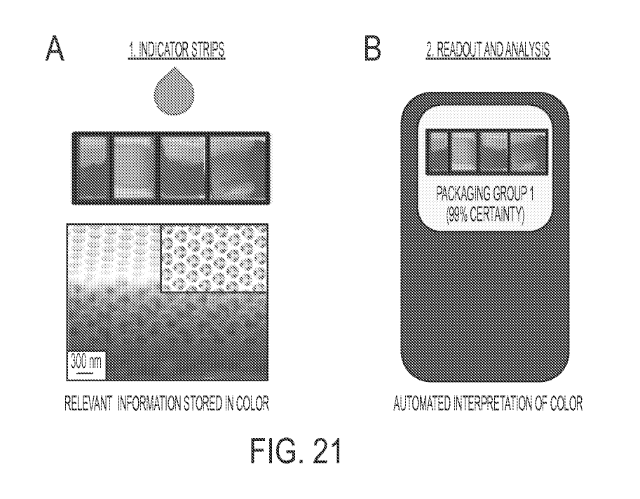

In some embodiments, software such as, image analysis, statistical analysis, comparison with stored calibration curves etc., can be used to analyze these signatures and rapidly print out a meaningful response to the user. This technique facilitates efficiently extracting drying stage information from optical signature using low-cost machine readouts. FIG. 21 shows a schematic of the automated readout concept using the IOF indicator strip. FIG. 21A shows IOF indicator strips that are exposed to liquid. This exposure generates complex information about their compositional profile which is observable through the optical signature of the indicator or indicator array. FIG. 21B shows a portable electronic device, such as a smartphone, that captures the static and/or dynamic optical signature of the indicator array. Software rapidly analyzes the signature and prints out the user-relevant information on the screen.

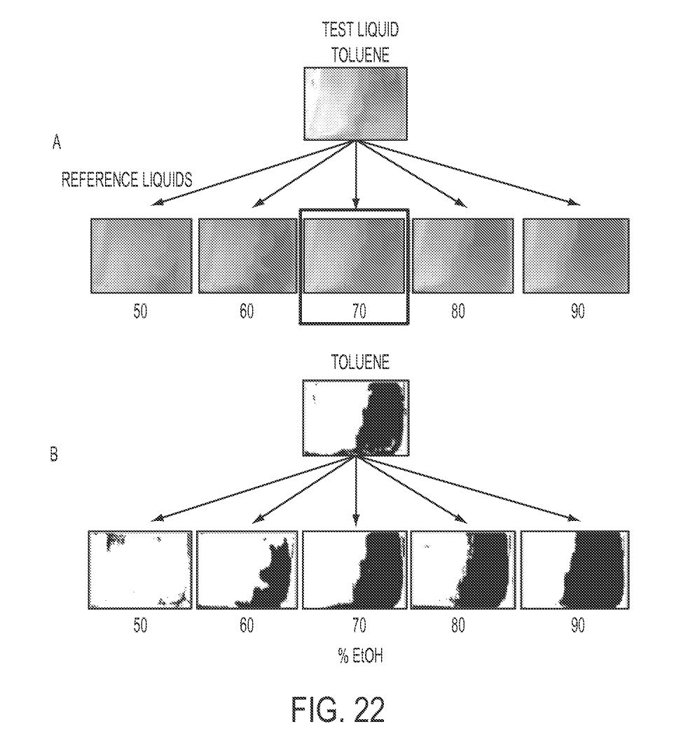

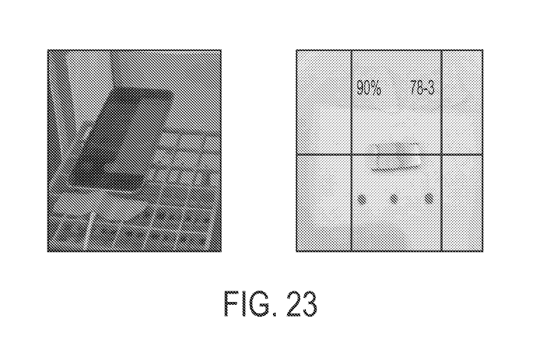

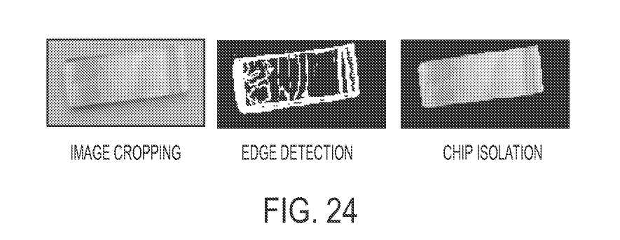

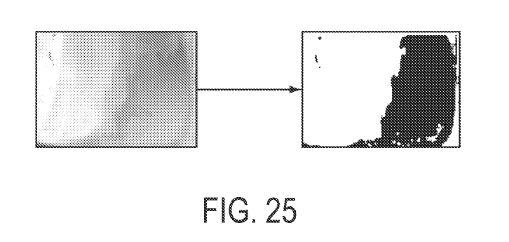

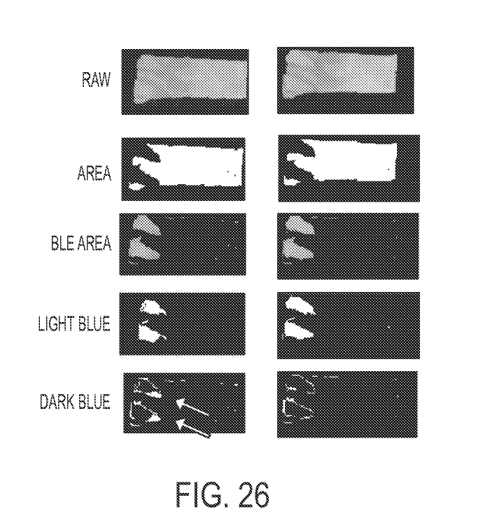

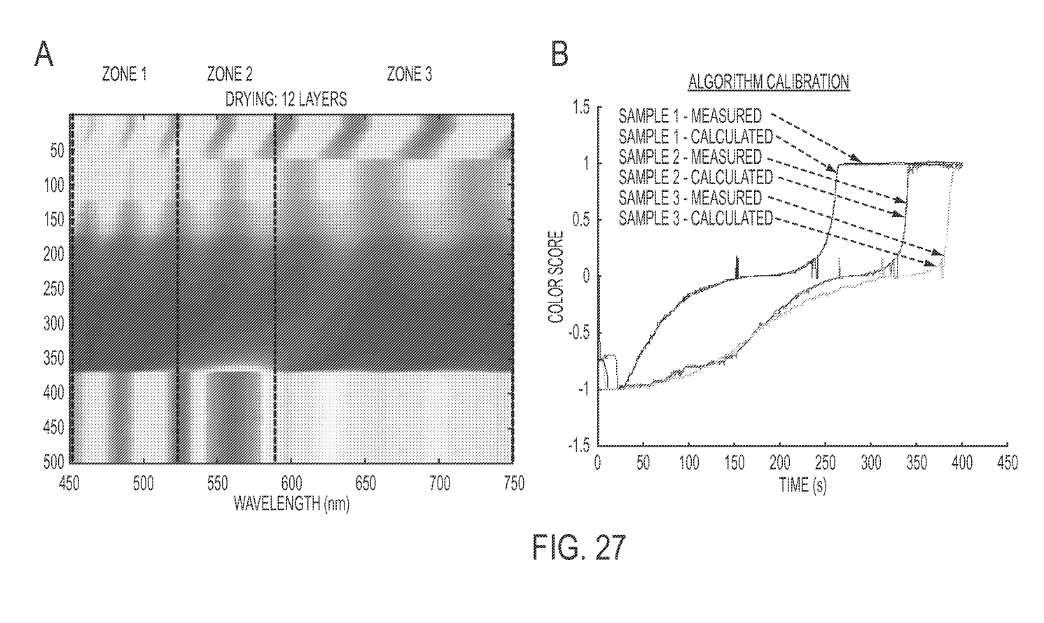

In some embodiments, several methods of detection that are compatible with a portable device can be used to determine the optical signature from partial wetting and/or drying. These include measures of color, such as photography and image analysis as shown in FIG. 22, FIG. 23, FIG. 24, FIG. 25 and FIG. 26, total scattering, as shown in FIG. 4, angle-dependence of scattering, as shown in FIG. 3, or comparing relative scattering from only a few spectral regions or through different color filters, as shown in FIG. 27.

FIG. 22 shows the use of image analysis to extract colored areas in an IOF that has been exposed to a liquid. Subsequently, quantification based on fraction of colored area can be obtained. In some embodiments, a software can analyze a smartphone image of one or more IOFs and analyze IOFs response upon exposure to a liquid. For example, a test liquid's response can be compared to a series of reference liquids and scored according to which gives the closest match. In the example illustrated in FIG. 22 70% ethanol in is the closet match. This is executed by an image analysis algorithm that discerns between completely filled area and partially/fully unfilled area in a raw image, as shown in FIG. 22A, and compares the shapes, as shown in FIG. 22B to determine a match.

The various algorithm components that can be used to extract highly specific color information, that allows partial filling states (either from drying or partial wetting) to be deduced, are shown in FIG. 23, FIG. 24, FIG. 25, and FIG. 26. FIG. 23 shows a housing that normalizes lighting conditions. FIG. 24 shows the algorithm component used for image cropping, edge detection and chip isolation. FIG. 25 shows the algorithm component used for thresholding and area detection. FIG. 26 shown the algorithm component used for color and edge specific tiebreakers.

In some embodiments the drying stage is monitored for relative reflectance through 3 color filters. FIG. 27 shows the drying stage measured by relative reflected intensity from an IOF in three spectral regions. FIG. 27 A shows the time evolution of the reflectance spectrum during drying from an IOF with the boundaries of the three spectral regions denoted. FIG. 27B shows the calibration curves for three experiments comparing direct calculation of the drying stage based on total reflectance (using the entire time curve to assign values) with calculated drying stage based on only the relative intensities of the reflectance in each of the three spectral regions (using only data at the current time point to assign a score). The normalized color score (denoting the stage of drying) is assigned on a scale from −1 to 1, where 0 denotes the point of minimum total reflectance. Positive scores denote degree of total reflectance recovery after the minimum (where 1 denotes the total reflectance of a completely dry film) and negative scores denote the negative of the relative decrease in total reflectance (where −1 denotes the point where the overlayer has dewetted and the onset of pore emptying).

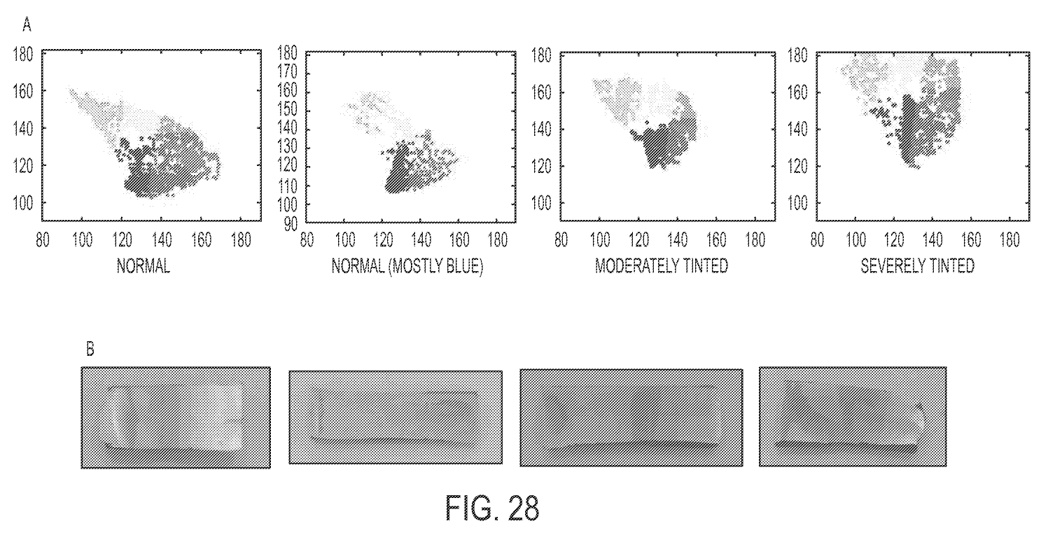

In some embodiments, the colorspace analysis enables color differentiation and accounting for colored liquids. FIG. 28 shows CIELAB color maps (top row) assigned to the IOF indicators wetted with non-transparent liquid, such as, crude oil, as illustrated here. By taking the severity of the tinting from crude oil more accurate color assignments have resulted. Since crude oil is not a clear liquid, it has the ability to shift the colors seen on IOF indicators. However, new code improvements detect the upward shift of the CIELAB color maps, as seen in FIG. 28A (top row), as crude oil increasingly tints the indicators, and adjusts the color assignments accordingly. Thus, what is now seen as gray is still labeled as blue, and so on, producing more accurate automated scores without affecting abnormally-wetting but normally-tinted samples, such as the mostly blue sample second from left.

### Examples of Using the System for Identifying Crude Oil Packaging Groups

As described herein, the W-Ink indicators can be used developed as a low-cost, portable, and easy-to-use indicators that will allow for rapid on-site identification of key physical properties of crude that are sufficient to determine its packaging group.

There has been a rapid growth over the last 10 years in the amount of crude oil shipped by rail in North America. This growth was stimulated by the discovery of large amounts of shale oil in the Bakken region of North Dakota. North American production now supplies 66% of U.S. crude oil demand, displacing crude from Latin America, Africa, and the Middle East^{[1]}. The result is undue stress on the logistics systems in place for transporting crude oil to domestic markets. Increasingly, crude oil transport by rail is favored as a quicker, more flexible alternative to new pipeline development. Railroads transported 423% more crude oil by volume from 2011 to 2012. The most current data available also determines rail transport consistently spills less crude oil per ton-mile over other modes of land transportation. However, safety concerns have been raised over high profile accidents involving crude oil transport via rail such as the tragic explosion and fire in Lac Megantic, Quebec in 2013.

The higher premium paid for rail transport of crude oil is offset by pricing discounts created by pipeline bottlenecks. In the face of an insufficient number of buyers, even railroad bottlenecks results in price discounting, making expediency in shipping crude oil of utmost priority for distributors. Therefore, in current practice, no testing is typically done to determine the chemical and physical properties of crude oil. Recently, a joint initiative by the Pipeline and Hazardous Materials Safety Administration (PHMSA) and the Federal Railroad Administration (FRA) called Operation Classification determined that 11 out of 18 randomly selected tanker cars were carrying crude oil that was misclassified. This discovery came after the PHMSA issued fines to several oil companies that did not comply with crude oil shipping classification standards. Blended from different sources wells before it is shipped, crude oil represents a highly variable mixture with respect to physical properties, such as flash point, and boiling point. This variability makes hazards difficult to predict and effective safe-handling procedures difficult to implement and the chemical properties of the oil remain unknown as it is pumped into tanker cars. The adoption of safe transportation protocols is challenging without this knowledge, but testing samples on a daily basis before shipping is costly and time-consuming for either the shippers or FRA inspectors. Ideally, a simple, low-cost diagnostic device that can estimate the composition of a crude sample and instantly determine its packaging group directly at the loading site would provide a convenient solution to this challenge.

Crude oil is classified into packaging groups based on the flash point and initial boiling point. Industry uses the ASTM D93 standard for evaluating the flash point of crude oil. ASTM D93 specifies the flash point as the lowest temperature corrected to a barometric pressure of 101.3 kPa, at which application of an ignition source causes vapors of a specimen of the sample to ignite under specified conditions of test. Similarly, industry uses the ASTM D86 standard for evaluation of the boiling point of crude oil. According to ASTM D86, the initial boiling pint is measured as the temperature at which the first droplet falls from the distillation column. Another standard used for the boiling point is the Reid vapor pressure.

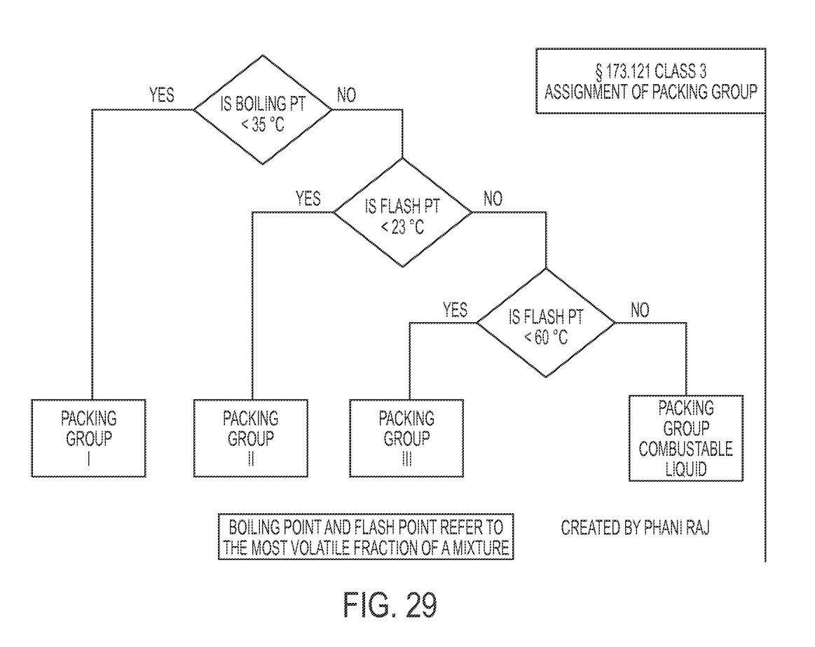

FIG. 29 shows packing group assignment for crude oil according to §173.121 class 3. The boiling point and flash point refer to the most volatile fraction of a mixture. When the boiling point is less than 35° C. the crude oil belongs to Packing Group I. When the boiling point is greater than 35° C. and the flash point is less than 23° C. the crude oil belongs to Packaging Group II. Similarly, when the flash point is greater than greater than equal to 23° C. but less than 60° C. the crude oil belongs to Packaging Group III. Additionally, when the flash point is greater than equal to 60° C., the crude oil belongs to the Packaging Group of combustible liquids. This is summarized below in Table 1.

TABLE 1
Packaging groups definition for crude oil

| Packing | | | | | |
|---|---|---|---|---|---|
| Group | Boiling Point | | Flash Point | | Examples |
| I | ≦35° C. | (95° F.) | | | Pentane |
| II | >35° C. | (95° F.) | <23° C. | (73° F.) | Gasoline, Hexane |
| III | >35° C. | (95° F.) | ≧23° C. | (73° F.), | Diesel, kerosene |
| | | | ≦60° C. | (140° F.) | |

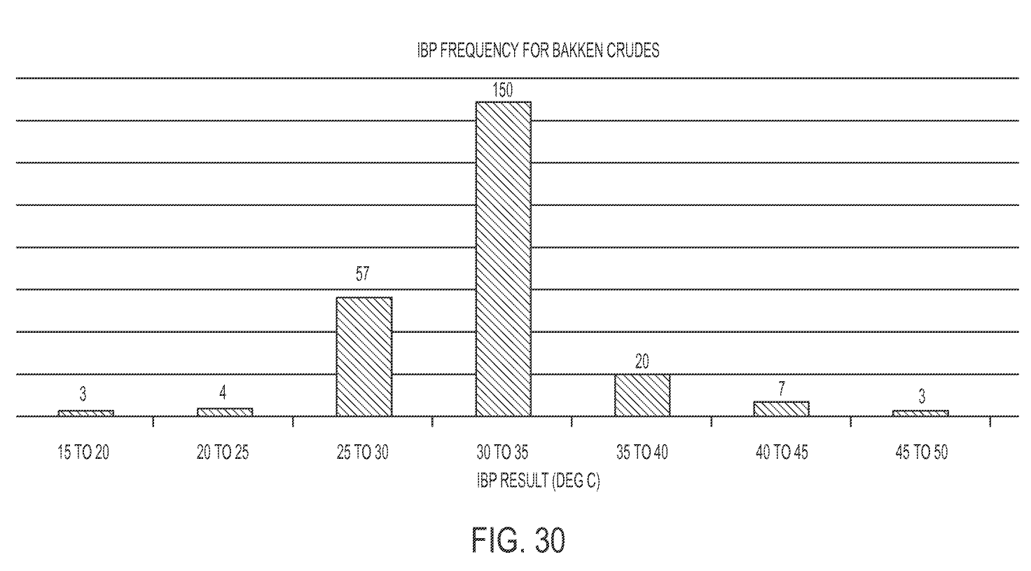

FIG. 30 shows the IBP frequency of Bakken crudes. It seen that IBP for most crude oils fell near the boundary between PG I and II.

Currently, an inexpensive diagnostic device for identifying packaging group of crude oil in the field is not available. Due to the explosion hazard associated with errors in identification of the packaging group of the crude oil in the field, there is a long felt need for a user-friendly, low-cost diagnostic device that can rapidly identify crude oil packaging group in the field. Such a devise will require little to no training or expertise and can be used by shippers of Federal Railroad Administration (FRA) inspectors directly on site.

The low-cost diagnostic device includes indicators that can operate based on W-ink, a colorimetric indicator technology, such as described in U.S. patent application Ser. No. 13/990,324, that displays visibly distinct color patterns in different liquids. These color patterns contain precise and detailed information about a liquid's wetting behavior against several different types of surfaces. Since wetting depends on both key physical properties (e.g. surface tension) and chemical properties (e.g. chemical interactions between the liquid and surface) of a liquid, the colorimetric readouts from W-Ink indicators can be used to extract enough information from a crude sample to identify the correct packaging group for its safe transportation.

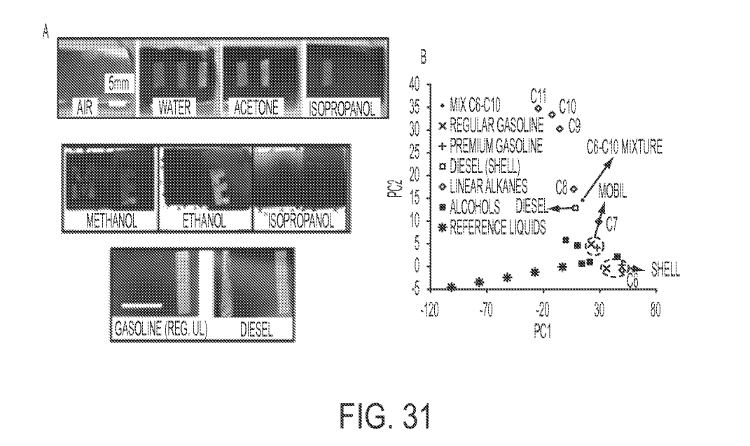

Colorimetric litmus tests such as pH paper are widely popular and commercially successful because of their low cost and ease of use. Structural color, exploiting photonic structures rather than molecular dyes, has the potential to greatly expand the applications of colorimetric indicators. Since color shifts in photonic structures are tied to changes in physical properties (e.g. size, shape, aspect ratio) rather than a specific chemical process, the optimization of the stimulus response and color effects can be more effectively decoupled. W-Ink and IOFs are colorimetric indicators for liquid identification that operate based on selective wetting in inverse-opal films. The regular porosity of these films causes them to display a highly selective threshold wettability for the onset of liquid infiltration. This pore geometry is also the source of iridescent color, a color that changes significantly when the pores fill with liquid due to refractive index changes. By generating spatial patterns in the structures' surface chemistry, the power to control the infiltration of different liquids to distinct and specific spatial patterns is realized. This facilitates an ability to project minute differences in liquids' wettability to macroscopically distinct, easy-to-visualize color patterns, as shown in FIG. 31A.

FIG. 31A shows W-ink indicators for colorimetrically distinguishing common clean room solvents (top), alcohols (middle), fuel types (bottom). FIG. 31B shows the numerated readout from an indicator array, illustrating the differentiation of a wide range of organic compounds, mixtures and petroleum products.

As wetting is a generic fluidic phenomenon, this indicator technology can be applied to distinguish liquids of any class, including petroleum products. To achieve increased chemical specificity, allowing meaningful differentiation of a greater number of compounds and even complex mixtures, a combinatorial measurement scheme is described herein, that includes an array of indicators that each use different chemistries to cover a redundant range of surface tensions, and a protocol to numerate the readout by comparing the colored area to that produced previously in a set of reference liquids (alcohol-water mixtures). Readouts for each array element were then categorized according to the reference liquid that produced the same color pattern, as shown in FIG. 31B. A computational algorithm that determined a liquid's identity from a library of possible unknowns based on these numerated color readouts was developed. Thus, this algorithm was able to automatically translate photographs of the color produced by the array into a liquid identity.

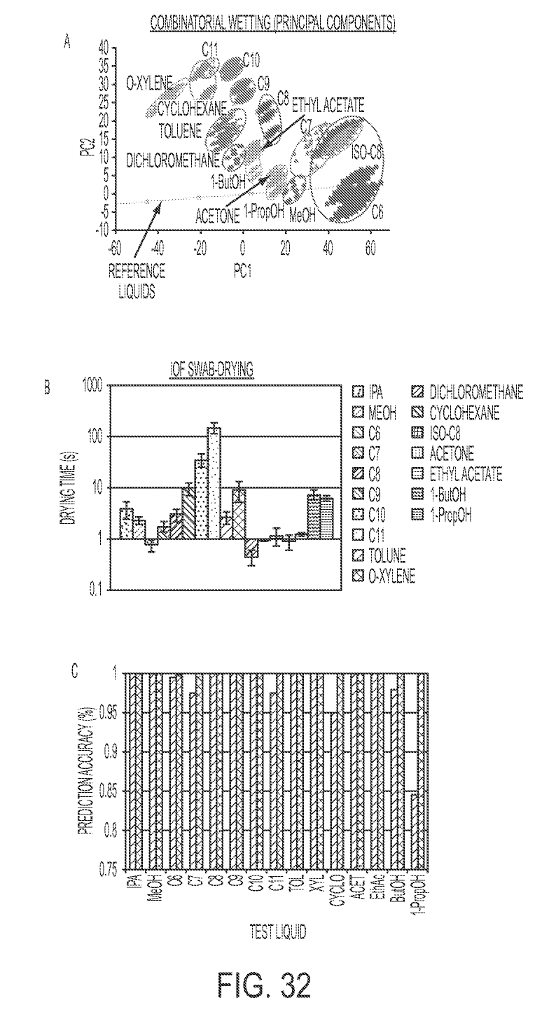

This method was capable of extracting a broad set of information about the physical and chemical properties of liquids and mixtures. For example, it identified common solvents with 98.6% accuracy from a library of 15 possible liquids, as shown in FIG. 32A. FIG. 32A shows the prediction accuracy analysis for a W-Ink array consisting of 6 chemical gradients. A principal component analysis algorithm was used to determine how well the liquids were differentiated. Comparing each reading to the data set excluding that point, this array identifies liquids with 98.6% accuracy from a library of 15 common solvents. FIG. 32B and FIG. 32C demonstrate that the mechanics of evaporation also couple strongly to color changes in W-Ink. This provides an extra independent dimension of information on the unknown liquids. Measurements of the time to the reappearance of color after swabbing with liquid, as shown in FIG. 32B, when combined with the information extracted from the wetting patterns FIG. 32A, increased the prediction accuracy to 99.99%, as shown in FIG. 32C. In tests that are similar to the identification of crude, this method was also capable of sorting alkanes by carbon chain length, and gasoline samples by their gas station of origin and type (e.g. gasoline vs. diesel). Monitoring the time scale of the reappearance of color due to evaporation further increases the specificity of the diagnostic device, as shown in FIG. 32B. The mechanics of evaporation also couple strongly to color changes in W-Ink, providing an extra independent dimension of information on the unknown liquids. When combined with the information extracted from the wetting patterns, as shown in FIG. 32A, increased the prediction accuracy to 99.99%, as shown in FIG. 32C. Since volatility is directly correlated with both physical properties that determine the packaging group for crude, this added dimension, if needed, is likely to produce highly accurate characterizations.

### Adaptation of Photonic Crystals for Rapid Determination of Crude Oil Packaging Group

As described herein, IOFs or W-Ink can be adapted to develop easy-to-use portable indicators for rapid identification of the packaging group for crude oil samples that can be used directly, either by the shipper or an FRA inspector directly on site. W-Ink may accurately sort petroleum liquids according their packaging group.

IOF or W-Ink indicator strips can be incorporated into a user-friendly field sampling kit, enabling reliable and safe collection of oil samples and fool-proof determination of the packaging group. Such a kit would include disposable W-Ink strips that are loaded onto a contraption that facilitates easy sampling and imaging, combined with simple imaging protocols and image analysis algorithms, performed on a portable device connected to the kit (e.g. smartphone or small electronic device) that would also give the user a clear report of the test result (e.g. a screen reading “PG I, 99.9% certainty”). Such a kit can enable rapid and completely automated determination of the relevant properties of an oil sample on site, by either the shipper or an FRA inspector, at a very low cost.

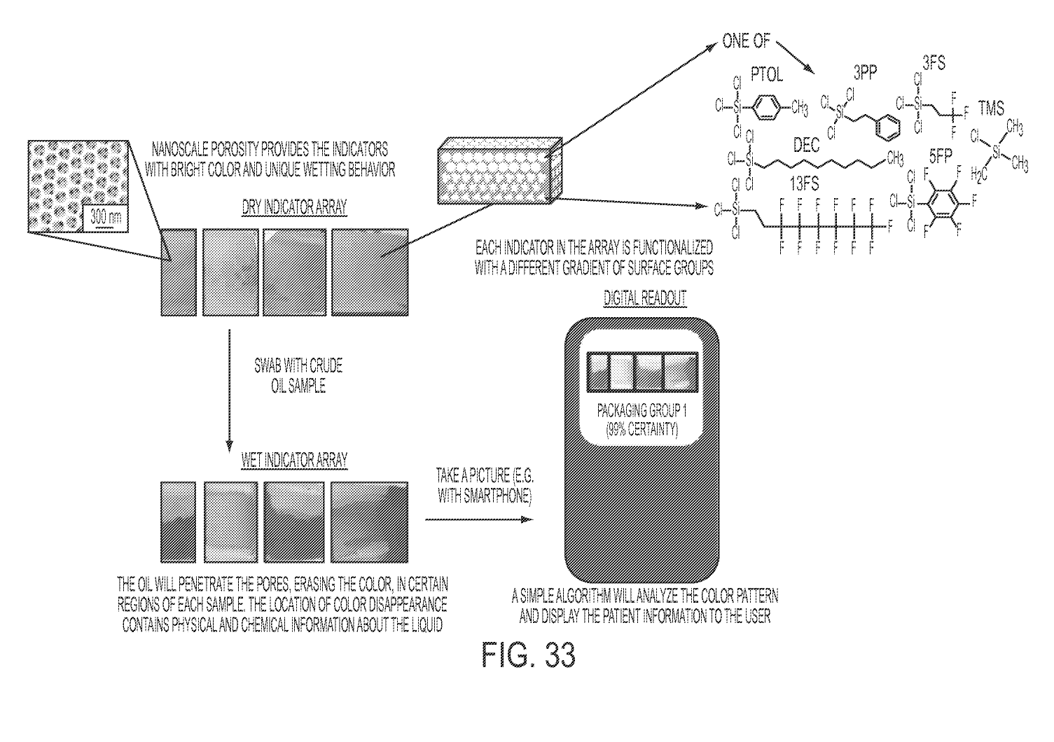

FIG. 33 shows a schematic of the system including an IOF or W-Ink indicator to be used for identification of the packaging group of crude oil with an accuracy greater than 95%. A dry IOF or W-Ink indicator array is shown in FIG. 33 A. The nanoscale porosity of the IOFs provide the indicators with bright color and unique wetting behavior. This is schematically shown in FIG. 33B. In some embodiments, each indicator in the array is functionalized with a different gradient of surface groups. This is shown schematically in FIG. 33C. When they are swabbed with a crude oil sample the wet indicator array is obtained as shown in FIG. 33D. Depending on the composition of the crude oil, the crude oil penetrates the pores, and in doing so, erases the color in certain specific regions of each sample. The location of color disappearance contains physical and chemical information about the crude oil sample. In some embodiments, the static and dynamic response to the exposure to the sample liquid, such as crude oil, is captured with a device, such as a smartphone. A simple algorithm can be used to analyze the color pattern and display the information regarding the sample liquid to the user. This is shown schematically in FIG. 33E, where the packaging group of the crude oil is displayed along with the accuracy of the identified result.

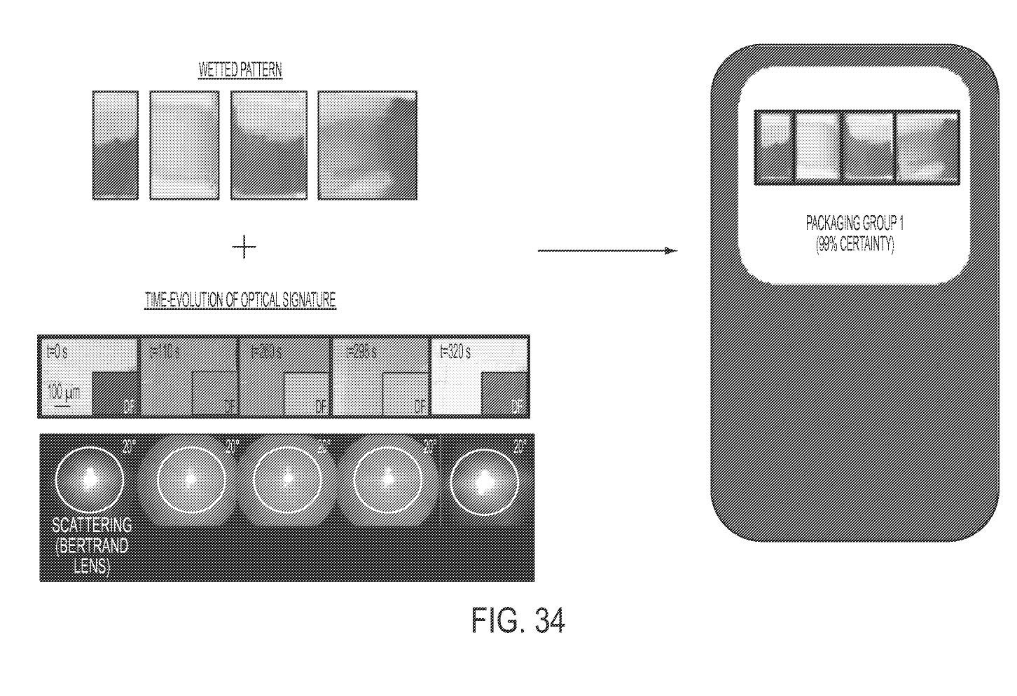

In some embodiment, the automated readout of wetting and drying of the IOF indicator arrays are used to extract compositional information about petroleum compounds and determine their transportation hazard classification. FIG. 34 shows a schematic illustrating a system using the automated readout of wetting and drying of the IOF indicator arrays for identifying information about petroleum compounds and ascertaining their hazard classification. An indicator or indicator array is exposed to the test liquid. The static/time-dependent optical signature is recorded and analyzed on a mobile device, which prints out the relevant information to the user (e.g. crude oil hazard class).

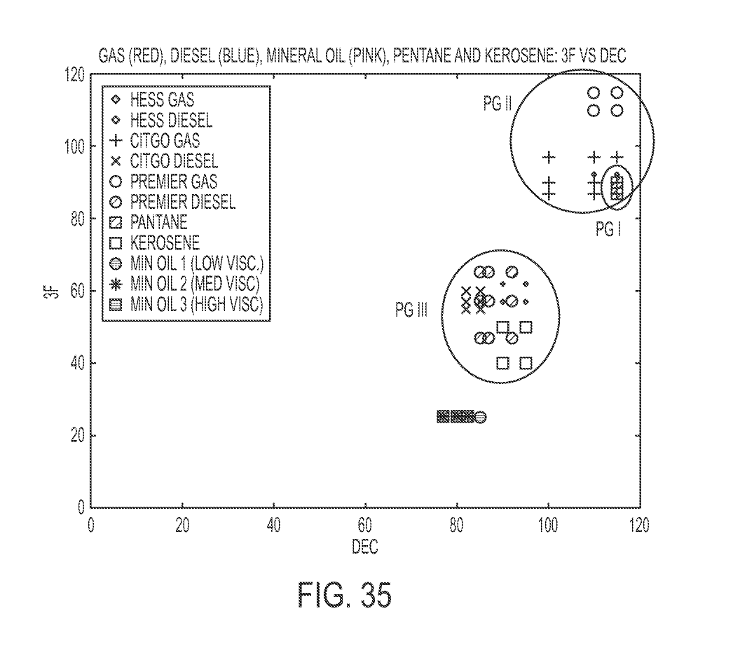

FIG. 35 shows the sorting of refined petroleum compounds using colorimetric wetting responses from two IOF indicators. Both indicators were functionalized with a vertical functionalization gradient with 1H,1H,2H,2H-tridecafuorooctyl-silyl (13F5) as the first functionality and with n-decyl-silyl (DEC) and 3,3,3-trifluoropropyl-silyl (3F) groups respectively as the second functionality. These indicators were then exposed to several different samples of gasoline (packaging group, PG II), diesel (PG III), pentane (PGI), kerosene (PG III), and mineral oils (all classified as Combustible Liquid) with three viscosities. Based on the exposure the system was able to identify the packaging group of each sample with a high degree of accuracy (low error rate).

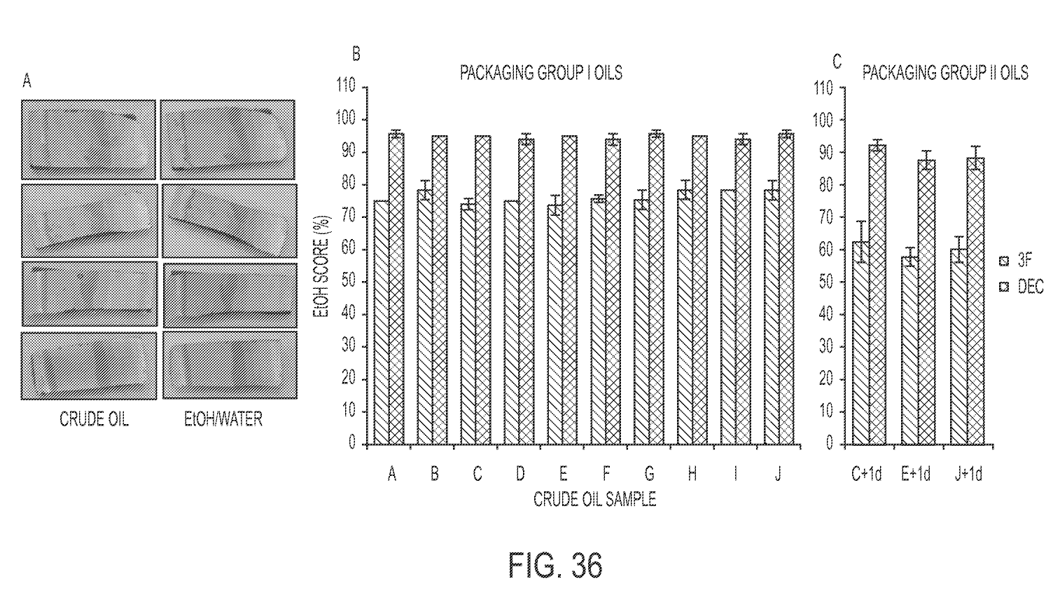

FIG. 36 shows the indication of packaging groups of crude oil samples using the same two IOF-indicator array used in FIG. 35. FIG. 36A shows the images of the indicators in ethanol/water mixtures (reference liquids) vs. in crude oil samples. This also illustrates that the effects of the liquid color that must be accounted for. FIG. 36B shows that similar responses were observed from 10 samples that were all categorized as Packaging Group I. FIG. 36C shows that after removal of the lightest fractions, which altered the samples to Packaging Group II samples, a significantly different response was observed.

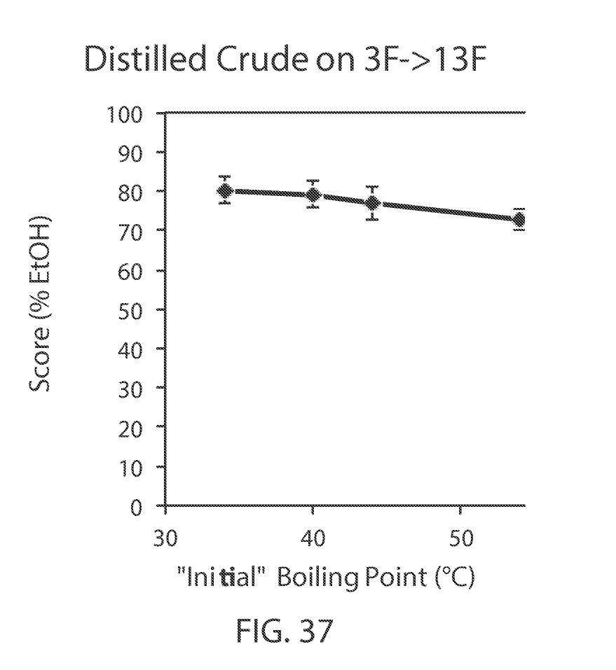

FIG. 37 shows the indicator response to initial boiling point of partially distilled crude oils. Here the 13FS→3F indicator response to various distillations of a crude oil sample as a function of initial boiling point shows that the score, i.e., match to the % EtOH, reduces as the initial boiling point increases.

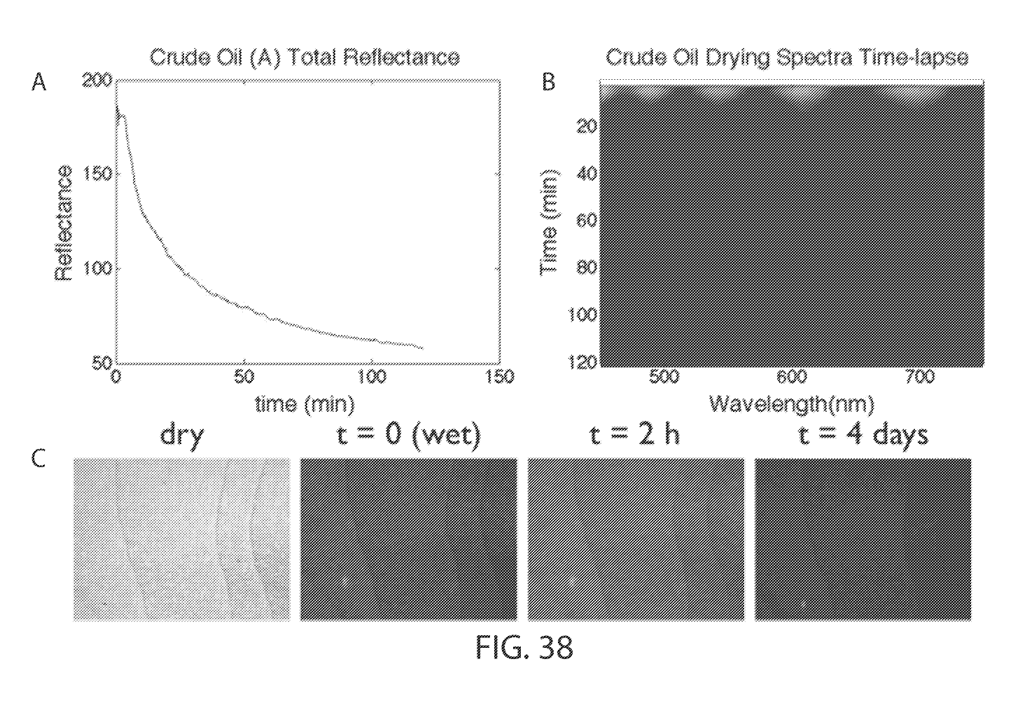

FIG. 38 shows the time-evolution of spectral signature during crude oil evaporation. FIG. 38A shows the time evolution of the total reflectance. FIG. 38B shows the time evolution of the reflectance spectrum. FIG. 38C shows the color as a sample of crude oil evaporates from the pores. The speed of spectral shift on different timescales reveals the different fractions of the volatility profile, with the longer-timescales reflecting the less volatile fractions.

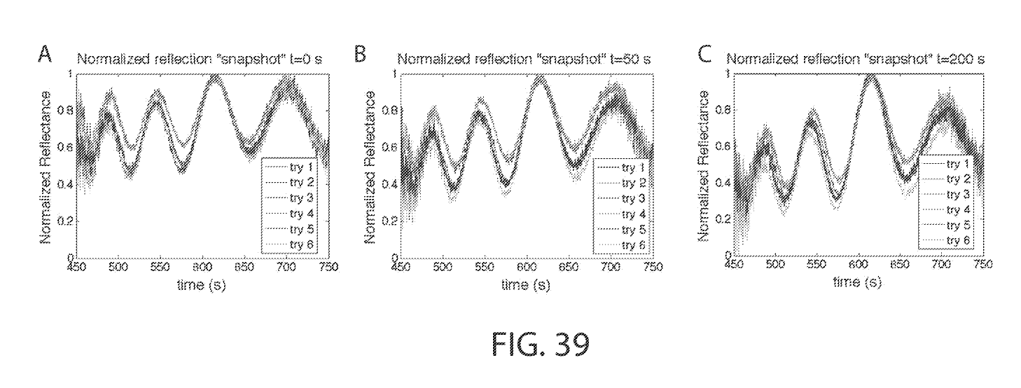

FIG. 39 shows the reproducibility of time-dependent optical response to crude oil during evaporation. Snapshots of the reflectance spectrum (normalized) from 6 different tests on a crude oil sample taken at 0 s, as shown in FIG. 39A, 50 s, as shown in FIG. 39B, and 200 s, as shown in FIG. 39C, show the reproducibility of the signature.

### Examples of Using the System for Detecting Prior Stimuli

As described herein, a tamper-indicating IOF can be developed that can be used to detect and record history of exposures to specific stimuli such as, exposure to moisture, pH, light, heat, oxygen etc.

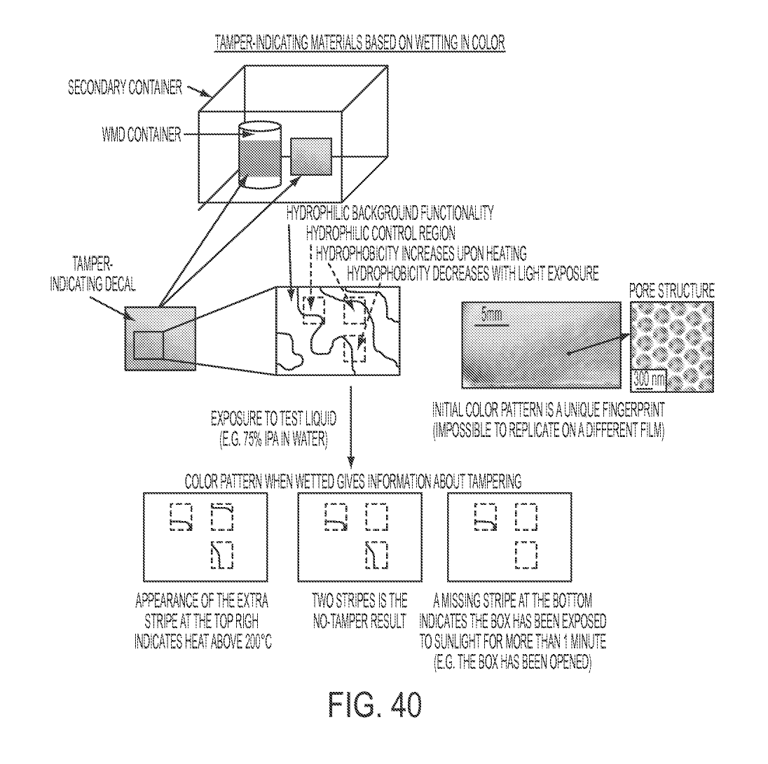

FIG. 40 shows a schematic of how the system can used to detect history of exposures to several stimuli. The IOF or W-Ink indicators can be placed either outside or inside of a container or secondary container holding a device, on the walls or door of a building housing, on cameras monitoring the devices of interest, etc. When dry, the wettability patterns are not visible, and films are characterized by color fingerprints (owing to varying total thickness) that are unique and impossible to forge. Information about different tamper stimuli is encoded in wettability responses of different regions. Exposure of the film to a specific decoding liquid (e.g. a specific concentration of water in rubbing alcohol) reveals information whether the degree of exposure is above or below a particular threshold. Exemplary values have been used for illustration in the schematic. In some embodiments, probing with different liquids (e.g. different concentrations) can provide reports on different thresholds.

In some embodiments, the devices described herein can be successfully fabricated on decals, and in paints, and deposition onto different types of relevant packaging.

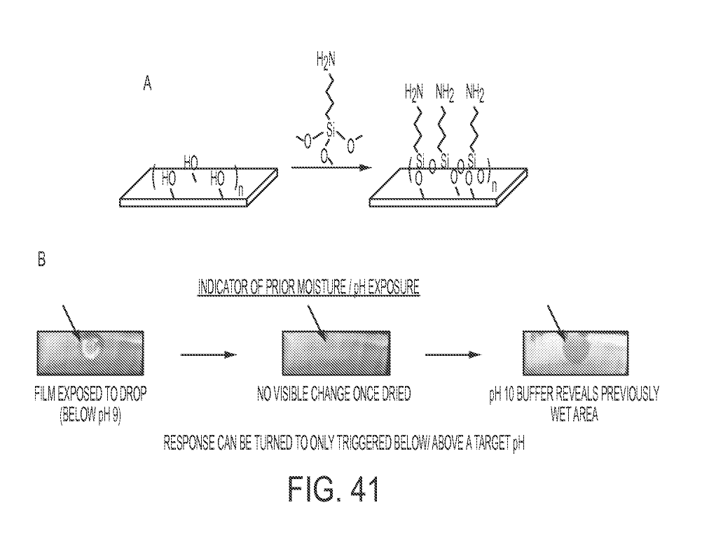

In some embodiments, the system can use used to detect moisture/pH exposure of a sample. FIG. 41 shows that the system can be used to detect previous exposure of the IOF indicators to liquids with specific pH values. In some embodiments, functionalization with amine-terminated surface groups, as shown in FIG. 41A, sensitizes the film to liquids with pH below the ionization pH of the surface groups. Immersion in a buffer reveals the areas previously wetted with liquids whose pH is less than the pH of the buffer solution. FIG. 41B illustrates that pH 10 buffer reveals areas previously wetted with liquids whose pH is less than 10. In different embodiments, surface groups can be modified to detect exposure above and below any target pH value (determined by the pKa of the surface group).

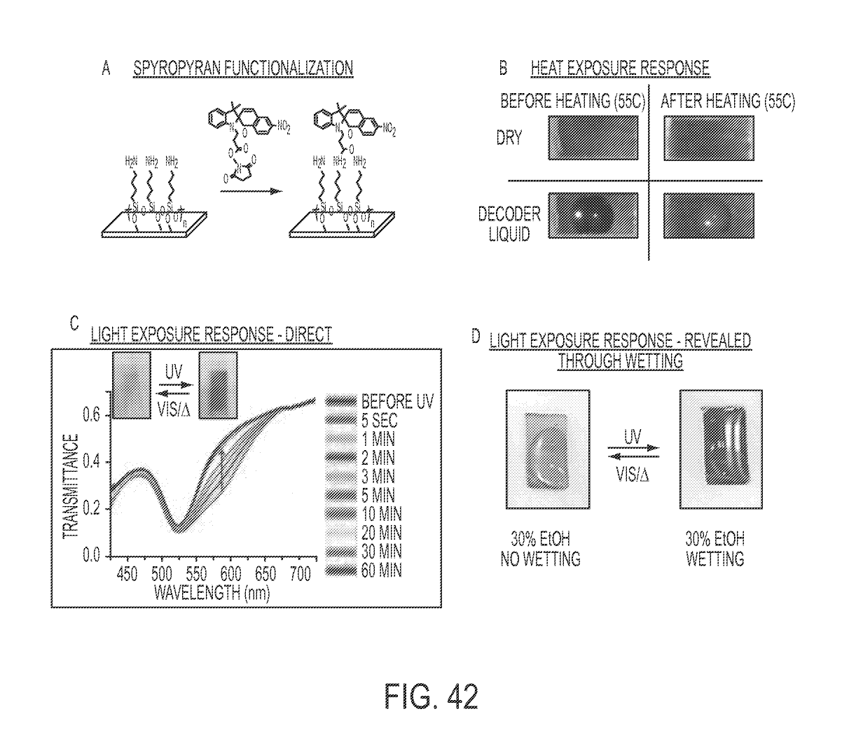

FIG. 42 shows that the system can be used to detect previous exposure of the IOF indicators to light or heat. Films functionalized with spyropyran-terminated surface groups, as shown in FIG. 42A, are modified in response to heat, as shown in FIG. 42B or light exposure, as shown in FIG. 42C and FIG. 42D. Modifications to the surface groups can be read out colorimetrically as changes in wetting behavior, as shown in FIG. 42B and FIG. 42D, or directly via color changes due to molecular absorption from the surface groups, as shown in FIG. 42C.

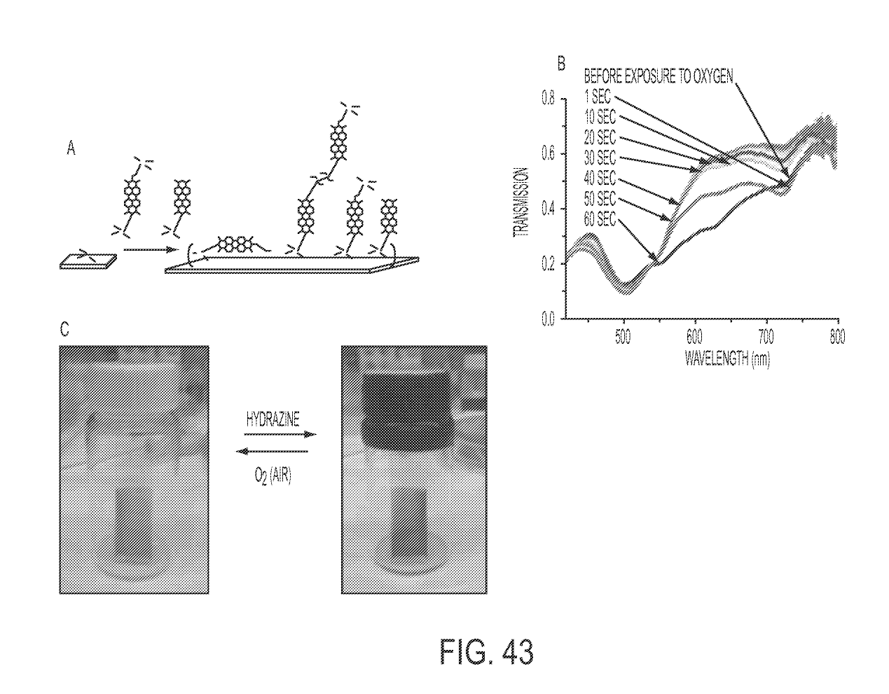

FIG. 43 shows that the system can be used to detect previous exposure of the IOF indicators to oxygen. In some embodiments, films functionalized with perylene diimide surface groups, as shown in FIG. 43A are modified through exposure to oxygen. This can be read out via direct color change, as shown in FIG. 43B and FIG. 43C or via wetting contrast. FIG. 43B shows the transmission spectrum changes of an inverse opal on glass substrate functionalized with negatively charged PDI upon exposure to air. FIG. 43C shows colorimetric response to oxygen of PDI functionanlized inverse opal on glass substrate. Left—PDI in a neutral state after exposure to air. Right—negatively charged PDI (through reduction with hydrazine vapors); the sample kept in oxygen free container.

Engineering stimuli-responsive surface chemistry is a promising route to detecting several different types of tampering. Changes in surface chemistry have the added advantage that they are generally optically undetectable when dry, making possible covert tamper indication. Below are some examples of tamper indication via surface chemistry that can be implemented.

### Photo-Responsive Surface Chemistry:

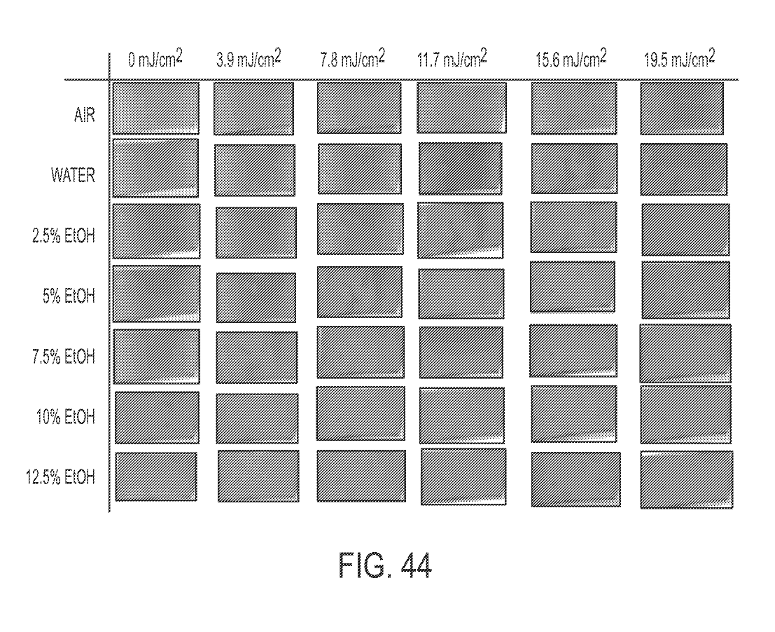

A tamper-indicating IOF whose surface chemistry can undergo irreversible changes when exposed to light can be developed. There are several well-known classes of materials that undergo light-induced changes in surface energy. For example, photo-induced cis-trans isomerization in azobenzene-containing surfaces provide a reversible means to record an exposure event. Photo-induced changes in water contact angles have been measured previously on azo-functionalized flat surfaces. Changes of 12° induced by a dose of ˜2 J/cm² of blue light have been observed. This required exposure level, sufficiently low to be inducible by sunlight, makes azo-containing surface groups attractive for recording of optical tamper stimuli (e.g. that could occur when a closed container is opened). The stability of these optically induced changes are adjustable by modifying the structure of the azo-containing moiety. Functionalized the surfaces of IOFs with the azo-containing polyelectrolyte, poly (Disperse Red 1)-co-(acrylic acid) can be used for detection of exposure to light. IOF films exhibit irreversible optically induced wettability changes due to photodegradation (bleaching) of the choromophore. This response is shown in FIG. 44. FIG. 44 shows the Photo-responsive wettability in IOFs with a poly (Disperse Red 1)-co-(acrylic acid) surface functionality. The onset of infiltration occurs at increasingly smaller ethanol concentrations in water with increasing UV exposure. The wetting threshold tunes continuously with the exposure dose. Since photodegradation of the molecule drives the effect, rather than chromophore alignment or cis-trans isomerization, higher doses (on the order of 4 J/cm²) may be used to achieve actuation. By further optimizing the azo-containing surface modifier, photo-induced wettability changes that do not rely on chromophore degradation (i.e. either on chromophore alignment or cis-trans isomerization) can be observed. Moreover, such changes may be observable at relevant doses for sunlight exposure (e.g. <1 J/cm²).

### Contamination (Humidity):

A tamper-indicating IOF whose surface chemistry can undergo irreversible changes when exposed to humidity can be developed. The interaction of a foreign substance with a surface can alter its surface energy through adsorption or reaction. An irreversible chemical reaction between the surface chemistry and the contaminant may be desirable for indication of tampering. This type of indicator, if placed on the inside of a sealed container would change if the seal were compromised. Adsorption of trichlorosilanes on hydroxylated surfaces in the absence of moisture can produce wetting behavior displaying a sensitive and irreversible humidity response. The formation and quality of trichlorosilane-derived self-assembled monolayers are known to be highly humidity sensitive. Alkyltrichlorosilane deposition in the absence of humidity is known to produce incomplete, un-cross-linked monolayers that display higher surface energies (lower contact angles) than those formed in the presence of water. It has also been shown that exposing the films to water after deposition of the trichlorosilanes produces a measurable increase in the water contact angle. IOFs functionalized with alkyl-and perfluoroalkyl-trichlorosilanes in the absence of humidity can exhibit θ_{c} for decoding liquids that irreversibly increases upon the film's first contact with humidity. This will manifest in colorimetrically distinct outcomes for exposed and unexposed films for liquids having θ_{c} near θ_{c,crit}.

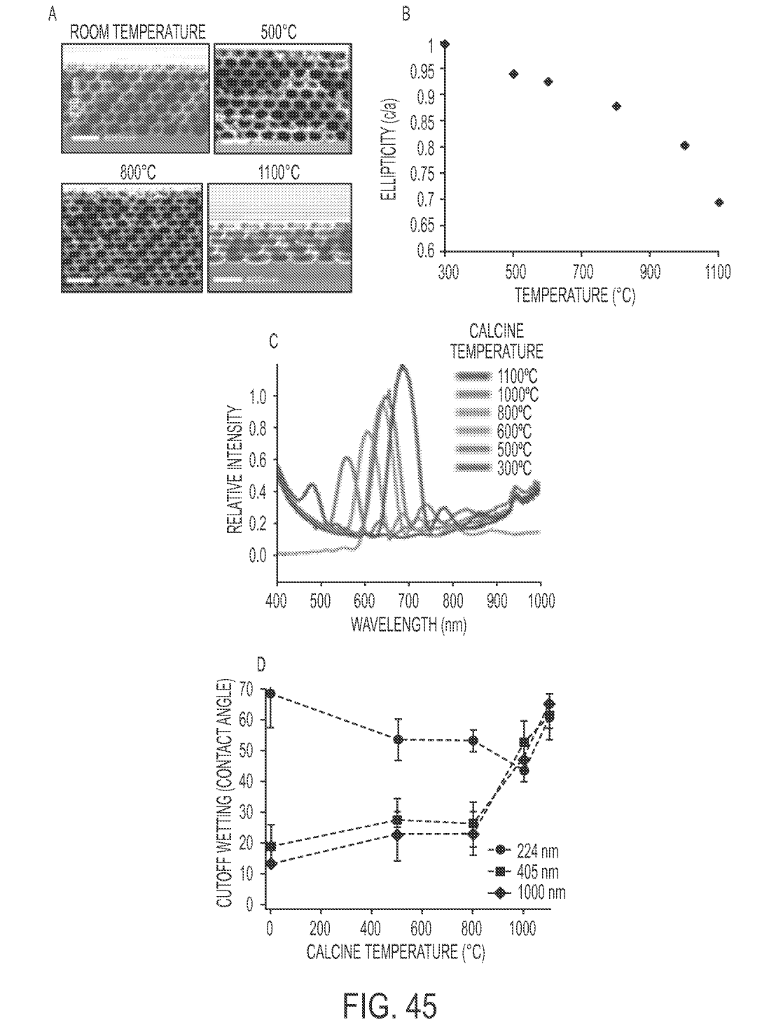

In some embodiments, IOFs whose pore geometry irreversibly changes in response to a tamper stimulus can display evidence of tampering through changes in both the wetting response and the dry color. Changes in the IOF pore geometry can be readily induced by thermal or mechanical stimuli. Experimental data shows that heating at 500° C. and above induces a significant temperature-dependent uniaxial compression on the lattice. This induces changes in both color and wetting behavior that are easy to detect. These results are summarized in FIG. 45. FIG. 45 shows the thermally and mechanically responsive wetting and color via pore collapse. FIG. 45A shows that IOFs made of SiO₂ displaying varying degrees of vertical collapse in response to heat treatment at different temperatures. FIG. 45B shows ellipticity (c/a) of the pores as a function of the maximum temperature to which the film was exposed. FIG. 45C shows changes in the dry color of an IOF as a function of heat treatment at different temperatures. FIG. 45C shows changes in the threshold value of θ_{c} as a function of temperature. This effect is caused by the shape of the inter-pore necks changing considerably, as the pores become increasingly asymmetric. This shrinkage of the pores affects the dry color of the IOFs by shrinking the vertical lattice-plane spacing and alters the wetting behavior by modifying the shape of the inter-pore necks. These results illustrate that mechanical strain on the pore structure also significantly affects the color and wetting response and implies that mechanical forces on the lattice could induce the same effect. The effects of direct mechanical stress on the pore structure and wetting properties can be characterized. Once the degree of this response is characterized, further optimization to the most relevant range of stresses (e.g. ˜1 psi for touch or ˜1000 psi for hammer strikes) can be made by changing the matrix material within the wide range of polymer and inorganic materials in which IOFs can be fabricated via colloidal co-assembly.

Since both thermal and mechanical stimuli can induce the same nature of response in these films, a simple method can be devised to distinguish the two. A very simple combinatorial method provides an effective way to accomplish this. Two different materials (e.g. SiO₂ and polymethylmethacrylate) can be incorproated into two different regions (e.g. pixels) of the decal or paint. Since the two materials have different thermal and mechanical constants they respond to each stimulus to differing degrees. By observing the combination of the wettability changes of the two IOFs, it is possible to isolate the extent of each stimulus.

Those skilled in the art would readily appreciate that all parameters and configurations described herein are meant to be exemplary and that actual parameters and configurations will depend upon the specific application for which the systems and methods of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that the invention may be practiced otherwise than as specifically described. The present invention is directed to each individual feature, system, or method described herein. In addition, any combination of two or more such features, systems or methods, if such features, systems or methods are not mutually inconsistent, is included within the scope of the present invention.

1. A system to measure properties of a liquid, comprising:
a colorimetric sensor comprising a photonic structure having functional groups on at least some of the interior surfaces of the porous photonic structure;
the colorimetric sensor that displays a first signature upon exposure to a sample liquid that wets at least some of the interior surfaces of the porous photonic structure and displays a signature different from the first signature as the sample liquid evaporates from the liquid;
a device to capture changes in the signature of the colorimetric sensor as a function of time;
a memory to store data representing the changes in signature of the colorimetric sensor; and
a processing unit to analyze the data captured by the device.

2-3. (canceled)

4. The system according to claim 1, wherein, the liquid comprises at least two components and the different regions with said different functional groups of the photonic structure attract the components of the liquid differently.

5. The system according to claim 1, wherein, the signature is a hierarchical defect pattern visible as controlled disorder in the photonic crystal structure that changes as the sample liquid evaporates.

6. The system of claim 1, wherein the signature is color of the colorimetric sensor that changes as the sample liquid evaporates.

7-9. (canceled)

10. The system of claim 1, wherein the signature is reflectance that changes as the sample liquid evaporates.

11. The system a claim 1, wherein the signature is angular distribution of off-angle scattering that changes as the sample liquid evaporates.

12. The system according to claim 1, wherein, the photonic structure is an inverse opal structure, a mesoporous silica, a short range order structure exhibiting structural color, a quasicrystal, or mixtures thereof.

13. The system according to claim 1, wherein the functional groups include reactive groups, protecting groups, hydrophilic groups, lyophilic groups, lyophobic groups, nanoparticles, or mixtures thereof.

14-22. (canceled)

23. The system according to claim 1, wherein, the device, the memory and the processing unit are housed in a mobile telecommunication device.

24-26. (canceled)

27. The system according to claim 1, wherein the sample liquid is a crude oil or a distillate of crude oil.

28. The system according to claim 27, wherein, the output information regarding the crude oil is a packaging group.

29-30. (canceled)

31. The system according to claim 27, wherein the output information regarding the crude oil is volatility.

32-68. (canceled)
